SEQUENCE LISTING (28 sequences)

SEQ ID NO: 1 - DNA, 20 bp, Artificial
  Artificially synthesized primer sequence for RT-PCR
tggggttggtgttctcatct

SEQ ID NO: 2 - DNA, 23 bp, Artificial
  Artificially synthesized primer sequence for RT-PCR
acctggtcacaaaacatcttcag

SEQ ID NO: 3 - DNA, 20 bp, Artificial
  Artificially synthesized primer sequence for RT-PCR
tggaatctagccatgcctct

SEQ ID NO: 4 - DNA, 23 bp, Artificial
  Artificially synthesized primer sequence for RT-PCR
atcacatgactactcaggagggg

SEQ ID NO: 5 - DNA, 20 bp, Artificial
  Artificially synthesized primer sequence for RT-PCR
ctgaactccagctgccctac

SEQ ID NO: 6 - DNA, 23 bp, Artificial
  Artificially synthesized primer sequence for RT-PCR
ggagaacagaggataaaaggctc

SEQ ID NO: 7 - DNA, 20 bp, Artificial
  Artificially synthesized primer sequence for RT-PCR
atcgccatctacaggagcag

SEQ ID NO: 8 - DNA, 23 bp, Artificial
  Artificially synthesized primer sequence for RT-PCR
ccaggcatgtctttgtaggtaac

SEQ ID NO: 9 - DNA, 20 bp, Artificial
  Artificially synthesized primer sequence for RT-PCR
accttcttaggcctcctggt

SEQ ID NO: 10 - DNA, 22 bp, Artificial
  Artificially synthesized primer sequence for RT-PCR
tacacgattgtcctcacccttc

SEQ ID NO: 11 - DNA, 20 bp, Artificial
  Artificially synthesized primer sequence for RT-PCR
ctggtatgcgactgggatct

SEQ ID NO: 12 - DNA, 21 bp, Artificial
  Artificially synthesized primer sequence for RT-PCR
tgattgactcagcaatgcagg

SEQ ID NO: 13 - DNA, 20 bp, Artificial
  Artificially synthesized primer sequence for RT-PCR
tcctgcactgactcctgaga

SEQ ID NO: 14 - DNA, 23 bp, Artificial
  Artificially synthesized primer sequence for RT-PCR
gaatagaaacagaaaccccttgg

SEQ ID NO: 15 - DNA, 20 bp, Artificial
  Artificially synthesized primer sequence for RT-PCR
ccgaggtggataacctgaca

SEQ ID NO: 16 - DNA, 23 bp, Artificial
  Artificially synthesized primer sequence for RT-PCR
gaattggtcgaaaagtcctcaac

SEQ ID NO: 17 - DNA, 20 bp, Artificial
  Artificially synthesized primer sequence for RT-PCR
ctcacacattgcgaacaaca

SEQ ID NO: 18 - DNA, 23 bp, Artificial
  Artificially synthesized primer sequence for RT-PCR
atggtgcttttaagaagagagcc

SEQ ID NO: 19 - DNA, 20 bp, Artificial
  Artificially synthesized primer sequence for RT-PCR
gcctcacaaaagagccagag

SEQ ID NO: 20 - DNA, 23 bp, Artificial
  Artificially synthesized primer sequence for RT-PCR
gatccacattggtgttaccagtt

SEQ ID NO: 21 - DNA, 20 bp, Artificial
  Artificially synthesized primer sequence for RT-PCR
cgaccactttgtcaagctca

SEQ ID NO: 22 - DNA, 23 bp, Artificial
  Artificially synthesized primer sequence for RT-PCR
ggttgagcacagggtactttatt

SEQ ID NO: 23 - DNA, 22 bp, Artificial
  Artificially synthesized primer sequence for RT-PCR
tgacagcatgaggaaacaaatc

SEQ ID NO: 24 - DNA, 20 bp, Artificial
  Artificially synthesized primer sequence for RT-PCR
acgaccccaagaaatgagtg

SEQ ID NO: 25 - DNA, 20 bp, Artificial
  Artificially synthesized primer sequence for RT-PCR
gcattggtggaggaggatac

SEQ ID NO: 26 - DNA, 24 bp, Artificial
  Artificially synthesized primer sequence for RT-PCR
tgccattaacattctagctactgg

SEQ ID NO: 27 - DNA, 20 bp, Artificial
  Artificially synthesized primer sequence for northern hybridization
ggaaaattcggaagaagcaa

SEQ ID NO: 28 - DNA, 20 bp, Artificial
  Artificially synthesized primer sequence for northern hybridization
cgggatttgttcagttcagg

(19) United States
(12) Patent Application Publication
(10) Pub. No.: US 2005/0214836 A1
(43) Pub. Date: Sep. 29, 2005

(54) Method of diagnosing ovarian endometriosis

(75) Inventors: Yusuke Nakamura, Yokohama-shi (JP); Toyomasa Katagiri, Shinagawa-ku (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Oncotherapy Science, Inc., Kawasaki-shi (JP); The University of Tokyo, Bunkyo-ku (JP)

(21) Appl. No.: 11/069,673

(22) Filed: Feb. 28, 2005

Related U.S. Application Data
(63) Continuation-in-part of application No. PCT/JP03/10257, filed on Aug. 12, 2003.
(63) Continuation-in-part of application No. PCT/JP04/13718, filed on Sep. 14, 2004.
(60) Provisional application No. 60/407,365, filed on Aug. 30, 2002.
(60) Provisional application No. 60/450,920, filed on Feb. 28, 2003.
(60) Provisional application No. 60/505,572, filed on Sep. 24, 2003.

(51) Int. Cl. C12Q 1/68; G01N 33/574

(52) U.S. Cl. 435/6; 435/7.23

(57) ABSTRACT

Disclosed are methods of diagnosing ovarian endometriosis using differentially expressed genes. Ovarian endometriosis associated genes identified herein or their gene products are useful as a diagnostic markers for identifying or detecting ovarian endometriosis. Also disclosed are methods of screening compounds serving as agents for treating ovarian endometriosis, and methods of treating ovarian endometriosis and method or vaccinating a subject against ovarian endometriosis.

### CROSS-REFERENCES TO RELATED APPLICATIONS

This application claims the benefit of U.S. Provisional Application Ser. No. 60/407,365, filed Aug. 30, 2002; 60/450,920, filed Feb. 28, 2003; 60/505,572 filed Sep. 24, 2003; and priority to International Application Nos. PCT/JP2003/010257, filed Aug. 12, 2003; and PCT/JP2004/013718, filed Sep. 14, 2004, the contents of which are hereby incorporated by reference in its entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### FIELD OF THE INVENTION

The present invention relates to methods of diagnosing ovarian endometriosis, and kits used for the diagnosis. The invention also relates to an ovarian endometriosis reference expression profile. The invention further relates to methods of screening a compound that inhibits or enhances the expression or activity of an ovarian endometriosis-associated gene.

### BACKGROUND OF THE INVENTION

Endometriosis is a common gynecologic disorder, affecting at least 10% of women of reproductive age (Rice (2002) Ann. N.Y. Acad. Sci. 955: 343-352). Endometriosis is defined as the presence of endometrial glands and stroma outside the uterus. Its most common symptoms are progressive dysmenorrhea, dyspareunia, chronic pelvic pain and infertility. Endometriosis with ovarian cysts can be diagnosed through techniques such as ultrasonography or magnetic resonance image (MRI); however, endometriosis without ovarian cysts is difficult to diagnose without surgical invasion (Rice (2002) Ann. N.Y. Acad. Sci. 955: 343-52) since the symptoms mentioned above are not specific to endometriosis. For example, though the concentration of CA125, one of the tumor markers for ovarian carcinoma, is sometimes elevated in the sera of women with endometriosis, due to its low specificity (Evers et al. (1995) “Progress Management of Endometriosis” ed. Cautinho, Parthenon Publishing Groups, Camforth, 175-84), it is not a very useful marker for the disorder. Furthermore, for a patient whose chief complaint is dysmenorrhea who shows no physical abnormality, it is difficult to distinguish endometriosis from idiopathic dysmenorrhea.

Treatment for endometriosis commonly involves surgical resection and/or medication with hormonal agents, such as gonadotropin-releasing hormone (GNRH) agonists and androgens. However, since long-term medication is undesirable due to side effects such as menopausal disorders (hot flushes and stiff shoulders), genital bleeding, and bone demineralization, clinical control of endometriosis is often very difficult. Current methods for the treatment of the disease are also problematic in that they are associated with a high recurrence rate. Specifically, the recurrence rate five years after operation and medical treatment has been reported to be about 20% (Redwine (1991) Fertil. Steril. 56: 628-34) and as much as 53% (Waller and Shaw (1993) Fertil. Steril. 59: 511-5), respectively.

Various studies of endometriosis have revealed little in terms of the underlying genetic and pathophysiologic mechanisms due to following reasons: (i) epithelial cells of endometrial cysts often wither and peel off such that it is very difficult to obtain sufficient sample material; and (ii) the effect of contamination of the stromal cells under the usual sampling methods is substantial. To overcome these problems, the present inventors scraped and microdissected epithelial cells from the cysts and obtained them with high purity in a forgoing study (Jimbo et al. (1997) Am. J. Pathol. 150: 1173-8).

Endometriosis is similar to malignant tumors in some respects. For example, endometriotic cells can be both locally and distantly metastatic. In other words, endometriotic cells can adhere, invade and damage other tissues in a manner similar to cancerous cells. Thus, to elucidate the nature of endometriosis, the present inventors carried out cDNA microarray analysis (Arimoto et al. (2003) Int. J. Oncol. 22: 551-60). By comparing expression patterns between endometriotic tissues and corresponding eutopic endometria using genome-wide cDNA microarray analyses, the present inventors identified several genes that were commonly up-regulated in endometrial cysts.

Tissue factor pathway inhibitor-2 (TFPI-2), also known as placental protein 5 (PP5), is a serine proteinase inhibitor containing three (3) tandemly arranged Kunitz-type proteinase inhibitor domains that is homologous to tissue factor pathway inhibitor (Sprecher et al. (1994) Proc. Natl. Acad. Sci. USA 91: 3353-7; Rao et al. (1996) Arch. Biochem. Biophys. 335: 82-92; Miyagi et al. (1994) J. Biochem. 116: 939-42). The protein is constitutively secreted from cells of several endothelial cell types (Iino et al. (1998) Arterioscler. Thromb. Vasc. Biol. 18: 40-6) in three alternatively glycosylated isoforms of 27, 31 and 33 kDa (Rao et al. (1996) Arch. Biochem. Biophys. 335: 82-92). TFPI-2 is a strong inhibitor of plasmin as well as of trypsin, chymotrypsin, plasma kallikrein, cathepsin G, factor VIIa and factor XIa, but not urokinase-type plasminogen activator (uPA), tissue plasminogen activator or thrombin (Sprecher et al. (1994) Proc. Natl. Acad. Sci. USA 91: 3353-7; Rao et al. (1995) Arch. Biochem. Biophys. 319: 55-62; Rao et al. (1995) Arch. Biochem. Biophys. 317: 311-4; Rao et al. (1995) J. Invest. Dermatol. 104: 379-83; Peterson et al. (1996) Biochemistry 35: 266-72). Quantification of this gene using cell-conditioned-medium, extracellular matrix (ECM) and cytoplasmic fractions revealed that most of the proteins encoded by the gene are present in the ECM (Rao et al. (1996) Arch. Biochem. Biophys. 335: 82-92). Furthermore, Neaud et al. reported that TFPI-2 potentates hepatocyte growth factor-induced invasion of hepatocellular carcinoma cells on its own (Neaud et al. (2000) J. Biol. Chem. 275: 35565-9), whereas the expression of TFPI-2 was shown to inversely correlate during the progression of human glioma (Rao et al. (2001) Clin. Cancer Res. 7: 570-6). Various gynecological studies about TFPI-2 have been performed in terms of the progression of pregnancy. However, to date, there is no report that TFPI-2 is associated with the development of endometriosis.

Intelectin (ITLN) had been reported as a secretory protein which might play a role in the recognition of bacterial-specific components in the host (Rao et al. (1996) Arch. Biochem. Biophys. 335: 82-92). Today, ITLN is proven to be a human lectin, binding to galactofuranosyl residues in the presence of Ca²⁺ that recognizes bacterial arabinogalactan of Nocardia containing D-galactofuranosyl residues (Tsuji et al. (2001) J. Biol. Chem. 276: 23456-63). ITLN is a secretory glycoprotein consisting of 295 amino acids and N-linked oligosaccharides, and comprises a basic structural unit of a 120 kDa homotrimer in which 40 kDa polypeptides are bridged by disulfide bonds. However, little is known whether ITLN participates in the progression or maintenance of various gynecologic disorders or tumor disease.

Invasion is a characteristic feature of endometriosis. According to the transplantation theory, endometriosis develops from endometrial fragments that are retrogradely menstruated into the peritoneal cavity. In order to develop into endometriotic lesions, these fragments must attach to the subperitoneal space and invade by interactions with ECM proteins. The proteolytic pathway of invasion depends on the balance of a lot of components including serine proteases, such as uPA and plasmin, matrix metalloproteinases and protease inhibitors (Shapiro (1998) Curr. Opin. Cell Biol. 10: 602-8; Toi et al. (1998) Breast Cancer Res. Treat. 52: 113-24; Andreasen et al. (1997) Int. J. Cancer 72: 1-22). Although the role of ECM-associated TFPI-2 is unclear, since TFPI-2 is primarily found in ECM, TFPI-2 may be important in the regulation of matrix turnover by serine proteases. Moreover, Neaud et al. reported that TFPI-2 induces an invasive activity in three different human hepatocellular carcinoma cell lines and stable transfectants (Neaud et al. (2000) J. Biol. Chem. 275: 35565-9). On the contrary, it has been demonstrated that TFPI-2 strongly inhibits the in vitro invasion of highly invasive HT1080 cell line (Rao et al. (1998) Int. J. Cancer 76: 749-56) and that its expression inversely correlates during the progression of human glioma (Rao et al. (2001) Clin. Cancer Res. 7: 570-6). As reported by Neaud et al., TFPI-2 may have both an indirect anti-invasive effect and a direct pro-invasive effect. Other protease inhibitors, for example, plasminogen activator inhibitor-1, also share a dual effect (Deng et al. (1996) J. Cell Biol. 134: 1563-71). The interesting phenomenon that an endometriotic cell can be both metastatic and invasive while the disease remains a benign disorder may be explained by the fact that TFPI-2 may have such a dual effect and controls the invasion of endometriotic cells.

The potential mechanism that ITLN is involved in the pathophysiology of endometriosis might be as follows. One hypothesis is that bacterial infection participates in the development of endometriosis. It has been reported that ITLN recognizes galactofuranosyl residues of bacteria (Tsuji et al. (2001) J. Biol. Chem. 276: 23456-63). When the infection with galactofuranosyl residue containing microorganisms causes the progression of endometriosis, the expression of ITLN may be elevated as a result of the response to the infection. Furthermore, ITLN has been known to be involved in the autoimmune response of endometriosis. Dysfunction of the immune system has been implicated in the etiology of endometriosis (Giudice et al. (1998) J. Reprod. Med. 43: 252-62; Lebovic et al. (2001) Fertil. Steril. 75: 1-10). Multiple reports describe the occurrence of autoantibodies to endometrial antigens (reviewed in Bums and Schenken (1999) Clin. Obest. Gynecol. 42: 586-610). Like ITLN, most of the autoantigens are glycoproteins. Thus, ITLN might act as an autoantigen of endometriosis. On the other hand, Lang and Yeaman reported that jacalin, a jackfruit lectin specifically binding to the Thomsen-Friedenreich antigen (Galp1-3GalNAc), binds to various endometriosis-associated autoantigens to consequently remove antibody reactivity with these autoantigens (Lang and Yeaman (2001) J. Autoimmun. 16: 151-61). There is another possibility that ITLN, like lectin jacalin, competitively works on the autoantigens and suppresses autoimmune response in endometriosis.

cDNA microarray technologies have facilitated the construction of comprehensive profiles of gene expression in normal and malignant cells, and the comparison of gene expression in malignant and corresponding normal cells (Okabe et al. (2001) Cancer Res. 61:2129-37; Kitahara et al. (2001) Cancer Res. 61: 3544-9; Lin et al. (2002) Oncogene 21:4120-8; Hasegawa et al. (2002) Cancer Res. 62:7012-7). This approach enables the disclosure of the complex nature of cancer cells, and assists in understanding the mechanism of carcinogenesis. Identification of genes that are deregulated in tumors can lead to more precise and accurate diagnosis of individual cancers as well as the development of novel therapeutic targets (Bienz and Clevers (2000) Cell 103:311-20). Medical applications of microarray technologies include: (i) discovery of genes that contribute to tumorigenesis; (ii) discovery of useful diagnostic biomarker(s) and novel molecular target(s) for anti-cancer agents; and (iii) identification of genes involved in conferring chemosensitivity. Recently, molecules associated with the development of certain types of cancers identified by microarray technologies have been clinically proven to be good targets for developing effective novel drugs for cancers. In an effort to discover mechanisms underlying tumors from a genome-wide point of view and target molecules for diagnosis and development of novel therapeutic agents, the present inventors have used a microarray of cDNAs representing 23,040 human genes to analyze expression profiles of tumors from various tissues (Okabe et al. (2001) Cancer Res. 61: 2129-37; Hasegawa S. et al. (2002) Cancer Res. 62: 7012-7; Kaneta et al. (2002) Jpn. J. Cancer Res. 93: 849-56; Kitahara et al. (2002) Neoplasia 4: 295-303; Lin et al. (2002) Oncogene 21: 4120-8; Nagayama S. et al. (2002) Cancer Res. 62: 5859-66; Okutsu et al. (2002) Mol. Cancer Ther. 1: 1035-42; Kikuchi et al. (2003) Oncogene 22: 2192-205). For example, by analyzing expression profiles of hepatocellular carcinomas (HCC), the present inventors discovered VANGLI, a gene frequently up-regulated in tumor cells, and demonstrated that suppression of its expression with antisense-oligonucleotides significantly decreased the growth of HCC cells and induced apoptotic cell death (Yagyu et al. (2002) Int. J. Oncol. 220: 1173-8).

Studies designed to reveal mechanisms of carcinogenesis have already facilitated identification of molecular targets for anti-tumor agents. For example, inhibitors of farnesyltransferase (FTIs) which were originally developed to inhibit the growth-signaling pathway related to Ras, whose activation depends on posttranslational farnesylation, has been effective in treating Ras-dependent tumors in animal models (He et al. (1999) Cell 99: 335-45). Clinical trials on human using a combination or anti-cancer drugs and anti-HER2 monoclonal antibody, trastuzumab, have been conducted to antagonize the proto-oncogene receptor HER2/neu; and have been achieving improved clinical response and overall survival of breast-cancer patients (Lin et al. (2001) Cancer Res. 61: 6345-9). A tyrosine kinase inhibitor, STI-57 1, which selectively inactivates bcr-abl fusion proteins, has been developed to treat chronic myelogenous leukemias wherein constitutive activation of bcr-abl tyrosine kinase plays a crucial role in the transformation of leukocytes. Agents of these kinds are designed to suppress oncogenic activity of specific gene products (Fujita et al. (2001) Cancer Res. 61: 7722-6). Therefore, gene products commonly up-regulated in cancerous cells may serve as potential targets for developing novel anti-cancer agents.

It has been demonstrated that CD8+ cytotoxic T lymphocytes (CTLs) recognize epitope peptides derived from tumor-associated antigens (TAAs) presented on MHC Class I molecule, and lyse tumor cells. Since the discovery of MAGE family as the first example of TAAs, many other TAAs have been discovered using immunological approaches (Boon (1993) Int. J. Cancer 54: 177-80; Boon and van der Bruggen (1996) J. Exp. Med. 183: 725-9; van der Bruggen et al. (1991) Science 254: 1643-7; Brichard et al. (1993) J. Exp. Med. 178: 489-95; Kawakami et al. (1994) J. Exp. Med. 180: 347-52). Some of the discovered TAAs are now in the stage of clinical development as targets of immunotherapy. TAAs discovered so far include MAGE (van der Bruggen et al. (1991) Science 254: 1643-7), gp100 (Kawakami et al. (1994) J. Exp. Med. 180: 347-52), SART (Shichijo et al. (1998) J. Exp. Med. 187: 277-88) and NY-ESO-1 (Chen et al. (1997) Proc. Natl. Acad. Sci. USA 94: 1914-8). On the other hand, gene products which had been demonstrated to be specifically over-expressed in tumor cells, have been shown to be recognized as targets inducing cellular immune responses. Such gene products include p53 (Umano et al. (2001) Brit. J. Cancer 84: 1052-7), HER2/neu (Tanaka et al. (2001) Brit. J. Cancer 84: 94-9), CEA (Nukaya et al. (1999) Int. J. Cancer 80: 92-7) and so on.

In spite of significant progress in basic and clinical research concerning TAAs (Rosenbeg et al. (1998) Nature Med. 4: 321-7; Mukheiji et al. (1995) Proc. Natl. Acad. Sci. USA 92: 8078-82; Hu et al. (1996) Cancer Res. 56: 2479-83), only limited number of candidate TAAs for the treatment of adenocarcinomas, including colorectal cancer, are available. TAAs abundantly expressed in cancer cells, and at the same time which expression is restricted to cancer cells would be promising candidates as immunotherapeutic targets. Further, identification of new TAAs inducing potent and specific antitumor immune responses is expected to encourage clinical use of peptide vaccination strategy in various types of cancer (Boon and can der Bruggen (1996) J. Exp. Med. 183: 725-9; van der Bruggen et al. (1991) Science 254: 1643-7; Brichard et al. (1993) J. Exp. Med. 178: 489-95; Kawakami et al. (1994) J. Exp. Med. 180: 347-52; Shichijo et al. (1998) J. Exp. Med. 187: 277-88; Chen et al. (1997) Proc. Natl. Acad. Sci. USA 94: 1914-8; Harris (1996) J. Natl. Cancer Inst. 88: 1442-5; Butterfield et al. (1999) Cancer Res. 59: 3134-42; Vissers et al. (1999) Cancer Res. 59: 5554-9; van der Burg et al. (1996) J. Immunol. 156: 3308-14; Tanaka et al. (1997) Cancer Res. 57: 4465-8; Fujie et al. (1999) Int. J. Cancer 80: 169-72; Kikuchi et al. (1999) Int. J. Cancer 81: 459-66; Oiso et al. (1999) Int. J. Cancer 81: 387-94).

It has been repeatedly reported that peptide-stimulated peripheral blood mononuclear cells (PBMCs) from certain healthy donors produce significant levels of IFN-γ in response to the peptide, but rarely exert cytotoxicity against tumor cells in an HLA-A24 or -A0201 restricted manner in 51Cr-release assays (Kawano et al. (2000) Cancer Res. 60: 3550-8; Nishizaka et al. (2000) Cancer Res. 60: 4830-7; Tamura et al. (2001) Jpn. J. Cancer Res. 92: 762-7). However, both of HLA-A24 and HLA-A0201 are one of the popular HLA alleles in Japanese, as well as Caucasian (Date et al. (1996) Tissue Antigens 47: 93-101; Kondo et al. (1995) J. Immunol. 155: 4307-12; Kubo et al. (1994) J. Immunol. 152: 3913-24; Imanishi et al. (1992) Proceeding of the eleventh International Histocompatibility Workshop and Conference Oxford University Press, Oxford, 1065; Williams et al. (1997) Tissue Antigen 49: 129). Thus, antigenic peptides of carcinomas presented by these HLAs may be especially useful for the treatment of carcinomas among Japanese and Caucasian. Further, it is known that the induction of low-affinity CTL in vitro usually results from the use of peptide at a high concentration, generating a high level of specific peptide/MHC complexes on antigen presenting cells (APCs), which will effectively activate these CTL (Alexander-Miller et al. (1996) Proc. Natl. Acad. Sci. USA 93: 4102-7).

### BRIEF SUMMARY OF THE INVENTION

The present invention is based on the discovery that the expression of certain genes correlates with ovarian endometriosis.

Accordingly, the present invention provides a method of diagnosing endometriosis in a subject by determining the expression level of an ovarian endometriosis-associated gene or protein in a subject-derived biological sample, and then comparing the detected gene or protein expression level to a control level.

In the context of the present invention, the phrase “ovarian endometriosis-associated gene” refers to a gene that is characterized by an expression level that differs in a cell obtained from an ovarian endometrial cell compared to a normal cell. Herein, this phrase may also be sometimes referred to as “OEX nucleic acids”, “OEX polynucleotides” or “OEX associated genes”. The corresponding polypeptides encoded by the genes are referred to as “OEX polypeptides”, “OEX proteins”, or “OEX gene products”. The ovarian endometriosis-associated genes include OEX 1-242. Unless indicated otherwise, “OEX” refers to any of the sequences disclosed herein (OEX 1-242). The phrase “normal cell” indicates a cell obtained from ovarian or uterine tissue but not an endometrial cyst cell.

The phrase “control level” refers to a gene or protein expression level detected in a control sample and includes both a normal control level and an ovarian endometriosis control level. A control level may be a single expression pattern derived from a single reference population or may be derived from a plurality of expression patterns. For example, the control level can be a database of previously tested expression patterns. A normal healthy individual is one with no clinical symptoms of ovarian endometriosis and a normal control level includes an expression level of OEX nucleic acids or polypeptides detected in such a normal healthy individual or in a population of individuals known not to suffer from ovarian endometriosis. For the comparison purposes, the normal control level is typically based on, for example, the expression level of OEX nucleic acids or polypeptides in the normal healthy subject. Based on this normal control level, a permissible range is set, for example, to be the standard value ±2 S.D. The method of setting up a normal control level and permissible range based on measured values of a diagnostic marker protein is well-known in the art. For example, the normal control level of an OEX nucleic acid or polypeptide of the present invention is less than 20 ng/mL serum, preferably less than 10 ng/mL serum, more preferably less than 5 ng/mL serum. On the other hand, an ovarian endometriosis control level comprises an expression level of an OEX nucleic acid or polypeptide detected in a patient diagnosed with ovarian endometriosis.

An alteration, e.g., an increase in the expression level of OEX nucleic acids, OEX 1-97 and 186-242 or their gene products as compared to a control level, indicates that the subject suffers from or is at risk of developing ovarian endometriosis. Specifically, when the expression level in a subject sample is increased as compared to a normal control level, the subject is indicated as suffering from or is at risk of developing ovarian endometriosis. Furthermore, an increase or similarity in the expression level of the OEX nucleic acids or polypeptides as compared to an ovarian endometriosis control level indicates that the subject suffers from ovarian endometriosis. In contrast, a decrease in the expression level of OEX 98-185 detected in a subject sample compared to a normal control level indicates that the subject suffers from or is at risk of developing ovarian endometriosis.

A decrease or similarity in the expression level of OEX 1-97 and 186-242 or their proteins compared to an ovarian endometriosis control level indicates that the subject suffers from or is at risk of developing ovarian endometriosis. In contrast, an increase or similarity in the expression level of OEX 98-185 or their proteins compared to a normal control level indicates that said subject suffers from or is at risk of developing ovarian endometriosis.

According to the present invention, an expression level of an OEX nucleic acid or polypeptide may be determined as being altered when its expression level increases 10%, 25%, 50% or more as compared to the control level. Alternatively, the expression level of an OEX nucleic acid or polypeptide may be determined as being altered when its expression level increases 1, 2, 5 or more folds as compared to the control level. The expression level may be determined by detecting hybridization, e.g., on an array, of an ovarian endometriosis-associated gene probe to a gene transcript in the subject-derived biological sample.

The subject-derived biological sample used in the present invention may be any sample including, but not limited to, serum, blood and tissue samples obtained from a test subject, e.g., a patient known to or suspected of having ovarian endometriosis. Examples of blood-derived samples include serum and plasma. Tissue samples include an epithelial cell or an epithelial cell from an endometrial cyst.

The invention also provides an ovarian endometriosis reference expression profile of the expression level of two or more genes of OEX 1-242. Alternatively, the invention provides an ovarian endometriosis reference expression profile of the expression levels of two or more genes of OEX 1-97 and 186-242 or OEX 98-185.

The invention further provides methods of screening a compound that inhibits or enhances the expression or activity of an ovarian endometriosis-associated gene, OEX 1-242, by contacting with a test compound a test cell expressing an ovarian endometriosis-associated gene or a cell into which a vector comprising a reporter gene linked downstream of a transcriptional regulatory region of an ovarian endometriosis-associated gene has been introduced and determining the expression level of the ovarian endometriosis-associated gene. The test cell may be an epithelial cell such as those from an endometrial cyst. Compounds altering the expression level of an ovarian endometriosis-associated gene or reporter gene are expected to reduce the symptom of endometriosis. A decrease in the expression level of one or more genes of OEX 1-97 and OEX 186-242 or that of the reporter gene linked downstream of a transcriptional regulatory region of OEX 1-97 or OEX 186-242 compared to a normal control level of the gene indicates that the test compound is an inhibitor of the ovarian endometriosis-associated gene and are expected to reduce the symptom of endometriosis. Alternatively, an increase of the expression level of one or more genes of OEX 98-185 or that of the reporter gene linked downstream of a transcriptional regulatory region of OEX 98-185 compared to a normal control level of the gene indicates that the test compound is an enhancer of expression of the ovarian endometriosis-associated gene and are expected to reduce the symptom of endometriosis.

Furthermore, the present invention provides method of screening a compound that inhibits or enhances the expression of an ovarian endometriosis-associated gene, wherein a polypeptide encoded by an ovarian endometriosis-associated gene is contacted with a test compound and determining the binding activity of the compound and the gene or the biological activity of the polypeptide. Compounds altering the binding activity of the compound and the gene or the biological activity of the polypeptide are expected to reduce the symptom of endometriosis. A decrease in the binding activity with or the biological activity of one or more polypeptides encoded by OEX 1-97 and OEX 186-242 compared to a normal control level of the gene indicates that the test compound is an inhibitor of the ovarian endometriosis-associated gene and is expected to reduce the symptom of endometriosis. Alternatively, an increase of the binding activity with or the biological activity of one or more polypeptides encoded by OEX 98-185 compared to a normal control level of the gene indicates that the test compound is an enhancer of the ovarian endometriosis-associated gene and is expected to reduce the symptom of endometriosis.

The present invention also provides a diagnostic kit comprising a detection reagent that binds to two or more OEX nucleic acids or that binds to a gene product encoded by the nucleic acid sequences. Also provided is an array of two or more nucleic acids that bind to OEX nucleic acids. Alternatively, a detection reagent binds to an OEX protein, preferably including an antibody that binds to an OEX protein. The kit may be used for the diagnosis of endometriosis according to the present invention.

Therapeutic methods of the present invention include methods of treating or preventing ovarian endometriosis in a subject by inhibiting the expression of a gene selected from the group of OEX 1-97 and OEX 186-242 or the activity of a polypeptide encoded by the gene. The method can be achieved, for example, by administering to the subject an antisense, short interfering RNA (siRNA), ribozyme or antibody composition.

The antisense composition reduces the expression of a specific target gene sequence. For example, the antisense composition contains a nucleotide sequence that is complementary to a coding sequence selected from the group consisting of OEX 1-97 and OEX 186-242. The siRNA composition reduces the expression of a nucleic acid sequence selected from the group consisting of OEX 1-97 and OEX 186-242. A nucleic acid-specific ribozyme composition may be constructed so as to reduce the expression of a nucleic acid sequence selected from the group consisting of OEX 1-97 and OEX 186-242.

Alternatively, the treatment or prevention of ovarian endometriosis in a subject is carried out by administering to said subject an antibody or fragment thereof that binds to a polypeptide encoded a gene selected from the group consisting of OEX 1-97 and 186-242.

The invention also includes vaccines and vaccination methods. For example, a method of treating or preventing ovarian endometriosis in a subject is carried out by administering to the subject a vaccine containing a polypeptide encoded by a nucleic acid selected from the group consisting of OEX 1-97 and 186-242 or an immunologically active fragment of such a polypeptide. An immunologically active fragment is, for example, a polypeptide that is shorter than the full-length of the naturally-occurring protein and which induces an immune response. For example, an immunologically active fragment of a length of at least 8 amino acid residues that stimulates immune cells, such as T cell or B cell, is encompassed by the immunologically active fragment of the present invention. Immune cell stimulation is measured by detecting cell proliferation, elaboration of cytokines (e.g., IL-2) or production of an antibody.

Other therapeutic methods include wherein a compound that increases the expression or activity of OEX 98-185 administered to the patient. Furthermore, ovarian endometriosis can be treated by administering a protein encoded by OEX 98-185. The protein may be directly administered to the patient or, alternatively, may be expressed in vivo subsequent to being introduced into the patient, for example, by administering an expression vector or host cell carrying the down-regulated marker gene of interest. Suitable mechanisms for in vivo expression of a gene of interest are known in the art.

Furthermore, the present invention provides compositions for treating or preventing ovarian endometriosis. The composition preferably comprises at least one active component selected from the group of (1) an antisense, siRNA or ribozyme against a gene selected from the group of OEX 1-97 and OEX 186-242; (2) an antibody or fragment thereof binding to a polypeptide encoded by a gene selected from the group of OEX 1-97 and OEX 186-242; (3) polynucleotide select from group consisting of OEX 98-185, or polypeptide encoded by thereof; and (4) a compound selected by any of the method of screening for a compound that alters the expression or activity of an ovarian endometriosis-associated gene of the present invention.

Unless otherwise defined, all technical and scientific terms used herein have the same meaning as commonly understood by one of ordinary skill in the art to which the present invention belongs. Although methods and materials similar or equivalent to those described herein can be used in the practice or testing of the present invention, suitable methods and materials are described below. All publications, patent applications, patents and other references mentioned herein are incorporated by reference in their entirety. In case of conflict, the present specification, including definitions, will control. In addition, the materials, methods and examples are illustrative only and not intended to be limiting.

Other features and advantages of the invention will be apparent from the following detailed description and from the claims.

### BRIEF DESCRIPTION OF THE DRAWINGS

The application file contains at least one drawing executed in color. Copies of this patent or patent application publication with color drawings will be provided by the Office upon request and payment of the necessary fee.

FIG. 1 depicts a photograph showing the results of cDNA microarray analysis. Expression of representative 10 genes and G3PDH was examined by semi-quantitative RT-PCR using cDNA prepared from amplified RNA. S, sample; and C, control. Symbols for genes whose function was known or inferred are noted; and Accession No. for ESTs.

FIG. 2a depicts a bar graph showing the Cy5/Cy3 intensity ratios of ALOX5AP in 23 endometrial cysts. The ratio was greater than 2.0 in all samples except No. 21.

FIG. 2b depicts a photograph showing the results of gene expression assay. The expression of ALOX5AP was up-regulated. G3PDH was used as an internal control.

FIG. 3 depicts the expression of TFPI-2 and ITLN in endometriosis. The expression of TFPI-2, ITLN and GAPDH was examined by semi-quantitative RT-PCR using cDNA prepared from amplified RNA. “C” indicates control. (A)The expression of TFPI-2 was up-regulated in 9 of 14 cases in the secretory phase; (B) the expression of ITLN was up-regulated in 10 of 14 cases in the secretory phase and in 4 of 9 cases in the proliferative phase.

FIG. 4 depicts the sub-cellular localization of the TFPI-2 and ITLN proteins. FIG. 4(A) depicts the results of Western blot analysis of myc-tagged TFPI-2 and ITLN proteins using extracts of COS-7 cells transfected with pcDNA3.1 -myc/His-sense plasmid or mock. “Cell” refers to cell lysate; and “Medium” refers to conditioned medium. Each conditioned medium included the corresponding protein. In addition, endogenous expression of TFPI-2 was detected in HEC-151 and Hs.683 cells. FIG. 4(B) depicts the results of immunofluorescent staining of TFPI-2-, ITLN-, and Mock-stable transfectants. FIG. 4(C) depicts the results of immunofluorescent staining of HEC-151 and Hs. 683 cells with anti-TFPI-2 antibody. Both exogenous and endogenous expression patterns of TFPI-2 were identical.

FIG. 5 depicts the results of Northern blot analysis of TFPI-2 and ITLN mRNAs. Multiple-tissue Northern blot membranes (Clontech) were hybridized with cDNA fragments of TFPI-2 or ITLN as described under the item “Materials and methods”. RNA size markers are indicated in kilobase pairs (kb).

FIG. 6 depicts the results of immunohistochemical staining of the TFPI-2 and ITLN proteins. FIG. 6(A) depicts the results of imunohistochemical staining of human adult normal tissue sections with anti-TFPI-2 antibody. Strong staining was observed in placenta, a much weaker staining in liver and heart, and no staining was observed in brain, kidney, lung and skeletal muscle. FIG. 6(B) depicts the results of immunohistochemical staining of human adult normal tissue sections with anti-ITLN antibody. Positive staining was observed in colon, much weaker in heart, and no staining in brain, kidney, liver and lung. FIG. 6(C) depicts the results of a cross-inhibition assay. Recombinant TFPI-2 protein inhibited TFPI-2 staining in placenta (upper panels). Similarly, recombinant ITLN protein inhibited ITLN staining in small intestine (lower panels).

FIG. 7 depicts the expression pattern of TFPI-2 and ITLN proteins in endometrial cysts examined by immunohistochemistry. Strong staining was observed for epithelial cells, and to some extent, stromal cells of endometrial cysts by anti-TFPI-2 antibody (left panels). On the other hand, positive staining was observed only on epithelial cells of the cysts by anti-ITLN antibody (middle panels). No positive staining could be observed in the same endometrial cysts tissues with anti-rabbit IgG as the negative control (right panels). Upper panels show images with lower magnification (X100) and lower panels show those with higher magnification (X200). Arrows indicate epithelial cells of the cysts.

FIG. 8(A) depicts the standard curve for TFPI-2 ELISA plotted using serially diluted recombinant TFPI-2. Each plotted point represents the mean value of duplicate measurements. FIG. 8(B) depicts a bar graph showing the result of detection of TRPI-2 in sera obtained from endometriosis patients and controls. TFPI-2 was detected in eight of 36 endometrial serum samples (p=0.023), in six of 21 rASRM stage IV samples (p=0.0097). No expression of TFPI-2 was detected in 20 control samples.

### DETAILED DESCRIPTION

The words “a”, “an” and “the” as used herein mean “at least one” unless otherwise specifically indicated.

To elucidate the nature of endometriosis, the present inventors carried out cDNA microarray analysis (Arimoto et al. (2003) Int. J. Oncol. 22: 551-60). By comparing expression patterns between endometriotic tissues and corresponding eutopic endometria using genome-wide cDNA microarray analysis, the inventors identified several genes that were commonly up-regulated in endometrial cysts. Two genes, encoding tissue factor pathway inhibitor-2 (TFPI-2) and intelectin (ITLN) were discovered among these up-regulated genes. These genes show relatively lower expression in normal human vital organ tissues and thus it is unlikely that this expression is essential for the maintenance or life. Therefore, suppressing the expression of these genes in an organism by targeting and such would not cause fatal side effects associated with the suppression of genes that are highly expressed in vital organs. In addition, both TFPI-2 and ITLN are known as secreted proteins. Detection of secreted protein is relatively easier as compared to non-secreted proteins because antibodies against the proteins and biological samples such as peripheral blood, which is easier to collect than tissues, can be used for the detection.

Therefore, the inventors contemplated that these proteins might function as diagnostic markers for endometriosis. The expression level of TFPI-2 in placenta is the highest in normal human tissues. While TFPI-2 circulates in blood of normal men and non-pregnant women in extremely low concentrations, its level increases 40-fold to 70-fold in the plasma of pregnant women (Buztow et al. (1988) Clin. Chem. 34: 1591-3). The expression of TFPI-2 in endometrial cysts indicates this protein may be secreted in the serum of endometriosis patients as well as in that of pregnant women. If this prediction is true, TFPI-2 may serve as a better marker for diagnosis of endometriosis. ITLN has been reported to be secreted in blood. Nevertheless, ITLN is a secretory protein which is suggested to circulate in blood of patients of endometriosis. Detection of these proteins in the serum of endometriosis patients was expected to establish a new diagnostic method for the disease.

Thus, in this study, cDNA microarray analysis was performed on over 20,000 genes. As a result, genes that were commonly over-expressed or suppressed (down-regulated or underexpressed) among ovarian endometriosis patients were selected. Two hundred forty-two genes were found to be differentially expressed in epithelial cells from endometrial cysts. Twenty-four genes were up-regulated throughout the menstrual cycle and thirty were down-regulated throughout the menstrual cycle. Seventy genes and ESTs were up-regulated during the proliferative phase of the menstrual cycle, and fourteen genes were down-regulated during the proliferative phase of the menstrual cycle. Sixty genes and ESTs were up-regulated during the secretory phase of the menstrual cycle, and forty-four genes were down-regulated during the proliferative phase of the menstrual cycle.

Furthermore, the present inventors established an enzyme-linked immunosorbent assay (ELISA) system to detect TFPI-2 protein in sera derived from patients with endometriosis, and demonstrated that the detected expression level of TFPI-2 protein was elevated in sera from patients of advanced stages of endometriosis among the examined sera. This result suggested that the production and secretion into serum of TFPI-2 protein increases in response to disease progress. Hitherto, CA125 was the most popular marker for endometriosis. However, as explained above, the specificity of CA125 as a marker for endometriosis is low and that of TFPI-2 protein seems to be higher, due to the detection of TFPI-2 only in sera from pregnant women and not in those from non-pregnant women and men. Accordingly, the findings of the present invention demonstrate that TFPI-2 protein is an effective marker for diagnosing endometriosis.

Hitherto, several hormonal therapies have been applied to the treatment of endometriosis. Endometriosis with ovarian chocolate cysts or severe adhesion is often treated by laparoscopic or abdominal surgery as well. However, these treatments are burdened by high recurrence rates and side effects.

In this study, the present inventors report that expression of these genes may play an important role in the progression or maintenance of endometriosis. Specifically, the present invention demonstrates the involvement of TFPI-2 protein in human ovarian endometriosis. Since the expression of this protein is relatively low in normal human adult tissues, gene encoding this protein has strong potential as target for therapy. Thus, the genes identified herein may be used for diagnostic purposes and these gene products are promising target for the development of drugs for inhibiting endometriosis and malignancies of ovarian and uterine tissue. By measuring expression of the various genes in a sample comprising a cell or population of cells, ovarian endometriosis can be diagnosed in a patient. Similarly, by measuring the expression of these genes in response to various agents, agents for treating ovarian endometriosis can be identified.

The genes whose expression levels are modulated (i.e., increased or decreased) in ovarian endometriosis patients are summarized in Tables 1-9. The genes have been previously described and are presented along with a database accession number.

TABLE 1
Genes up-regulated in endometrial cysts throughout the menstrual cycle

| | GenBank | | | |
|---|---|---|---|---|
| | Accession | | | OEX |
| ratio ≧ 2 | No. | Abbreviation | Gene name | Assignment |
| 22 | M63262 | ALOX5AP | arachidonate 5-lipoxygenase-activating protein | 1 |
| 22 | AA583491 | HCA112 | hepatocellular carcinoma-associated antigen | 2 |
| | | | 112 | |
| 21 | X00457 | HLA-DPA1 | major histocompatibility complex, class II, DP | 3 |
| | | | alpha 1 | |
| 20 | M77349 | TGFBI | transforming growth factor, beta-induced, 68 kD | 4 |
| 20 | K01505 | HLA-DQA1 | major histocompatibility complex, class II, DQ | 5 |
| | | | alpha 1 | |
| 20 | X63629 | CDH3 | cadherin 3, type 1, P-cadherin (placental) | 6 |
| 20 | D85376 | | Human DNA for thyrotropin-releasing hormone | 7 |
| | | | receptor, exon 3 and complete cds | |
| 19 | K01171 | HLA-DRA | major histocompatibility complex, class II, DR | 8 |
| | | | alpha | |
| 18 | X00637 | HP | haptoglobin | 9 |
| 18 | M81141 | HLA-DQB1 | major histocompatibility complex, class II, DQ | 10 |
| | | | beta 1 | |
| 18 | M86511 | CD14 | CD 14 antigen | 11 |
| 18 | M15178 | HLA-DRB1 | major histocompatibility complex, class II, DR | 12 |
| | | | beta 1 | |
| 17 | U70136 | PRG4 | proteoglycan 4, (megakaryocyte stimulating | 13 |
| | | | factor, articular superficial zone protein) | |

Other genes

| | | | |
|---|---|---|---|
| 21 | M32093 | ESTs | 14 |
| 17 | AI310156 | ESTs, Weakly similar to A4P_HUMAN | 15 |
| | | INTESTINAL MEMBRANE A4 PROTEIN | |
| | | [H. sapiens] | |

Genes with normalized expression ratios (cyst/normal) of ≧2.0 in more than 70% of the 23 cases were examined. The numbers of samples in that category are indicated in the left-most column. Accession numbers, gene symbols and names were retrieved from the Unigene Database (build#131).

TABLE 2
Genes up-regulated in endometrial cysts from patients
in the proliferative phase of the menstrual cycle

| | Genbank | | | |
|---|---|---|---|---|
| | Accession | | | OEX |
| ratio ≧ 2 | No. | Abbreviation | Gene name | Assignment |
| 9 | AA548449 | TPM1 | tropomyosin 1 (alpha) | 16 |
| 9 | AA778308 | RNASE1 | ribonuclease, RNase A family, 1 (pancreatic) | 17 |
| 8 | AA985222 | CTSB | cathepsin B | 18 |
| 8 | AA779820 | RBP1 | retinol-binding protein 1, cellular | 19 |
| 8 | AF034374 | | molybdenum cofactor biosynthesis protein | 20 |
| | | | A; molybdenum cofactor biosynthesis | |
| | | | protein C | |
| 8 | M83202 | LTF | lactotransferrin | 21 |
| 8 | H52870 | CDC10 | CDC10 (cell division cycle 10, S. cerevisiae, | 22 |
| | | | homolog) | |
| 8 | X06617 | RPS11 | ribosomal protein S11 | 23 |
| 8 | AF023462 | PHYH | phytanoyl-CoA hydroxylase (Refsum | 24 |
| | | | disease) | |
| 8 | AI287963 | PRP8 | U5 snRNP-specific protein (220 kD), | 25 |
| | | | ortholog of S. cerevisiae Prp8p | |
| 8 | X99920 | S100A13 | S100 calcium-binding protein A13 | 26 |
| 7 | V00478 | ACTB | actin, beta | 27 |
| 7 | U44403 | SLA | Src-like-adapter | 28 |
| 7 | U90913 | TIP1 | Tax interaction protein 1 | 29 |
| 7 | Y13287 | GDI2 | GDP dissociation inhibitor 2 | 30 |
| 7 | M55513 | KCNA5 | potassium voltage-gated channel, shaker- | 31 |
| | | | related subfamily, member 5 | |
| 7 | AA627679 | CHN2 | chimerin (chimaerin) 2 | 32 |
| 7 | AA921313 | RPL11 | ribosomal protein L11 | 33 |
| 7 | L12535 | RSU1 | Ras suppressor protein 1 | 34 |
| 7 | J04130 | SCYA4 | small inducible cytokine A4 (homologous to | 35 |
| | | | mouse Mipb) | |
| 7 | AA961412 | UBA52 | ubiquitin A-52 residue ribosomal protein | 36 |
| | | | fusion product 1 | |
| 7 | AA399392 | VPS11 | vacuolar protein sorting 11 (yeast homolog) | 37 |
| 7 | D16469 | ATP6S1 | ATPase, H+ transporting, lysosomal | 38 |
| | | | (vacuolar proton pump), subunit 1 | |
| 7 | AF006084 | ARPC1B | actin related protein 2/3 complex, subunit 1A | 39 |
| | | | (41 kD) | |
| 7 | X99209 | HRMT1L1 | HMT1 (hnRNP methyltransferase, S. | 40 |
| | | | cerevisiae)-like 1 | |
| 7 | AA434323 | P5CR2 | pyrroline 5-carboxylate reductase isoform | 41 |
| 7 | J02854 | MYRL2 | myosin regulatory light chain 2, smooth | 42 |
| | | | muscle isoform | |

Other genes

| | | | | |
|---|---|---|---|---|
| 8 | AI333234 | | hypothetical protein | 43 |
| 8 | AA703807 | | ESTs | 44 |
| 8 | R61506 | LOC51303 | FK506 binding protein precursor | 45 |
| 7 | H40445 | | ESTs, Weakly similar to pro alpha 1(I) | 46 |
| | | | collagen [H. sapiens] | |
| 7 | H12942 | | ESTs | 47 |
| 7 | AI038441 | | ESTs | 48 |
| 7 | AA994249 | | ESTs | 49 |
| 7 | AI242789 | | ESTs | 50 |
| 7 | AI222007 | | ESTs | 51 |
| 7 | AA921763 | | ESTs | 52 |
| 7 | BE894625 | | Homo sapiens cDNA clone IMAGE: 3918395 | 53 |
| | | | 5′ mRNA sequence | |
| 7 | AA421326 | | Homo sapiens cDNA: FLJ21918 fis, clone | 54 |
| | | | HEP04006 | |
| 7 | K00627 | | Human kpni repeat mRNA (cDNA clone | 55 |
| | | | pcd-kpni-8), 3′ end | |
| 7 | W79221 | PTD009 | PTD009 protein | 56 |
| 7 | AW513042 | KIAA1169 | two-pore channel 1, homolog | 57 |

Genes with normalized expression ratios (cyst/normal) of ≧2.0 in more than 70% of the nine cases were examined. The left-most column indicates the numbers of samples in that category. Accession numbers, gene symbols and names were retrieved from the Unigene Database (build#131).

TABLE 3
Genes up-regulated in endometrial cysts only during
the secretory phase of the menstrual cycle

| | Genbank | | | |
|---|---|---|---|---|
| | Accession | | | OEX |
| ratio ≧ 2 | No. | Abbreviation | Gene name | Assignment |
| 14 | W45244 | C3 | complement component 3 | 58 |
| 13 | D28124 | NBL1 | neuroblastoma, suppression of | 59 |
| | | | tumorigenicity 1 | |
| 13 | AA319695 | CEBPD | CCAAT/enhancer binding protein (C/EBP), | 60 |
| | | | delta | |
| 13 | X67698 | HE1 | epididymal secretory protein (19.5 kD) | 61 |
| 13 | S67310 | BF | B-factor, properdin | 62 |
| 13 | L42176 | FHL2 | four and a half LIM domains 2 | 63 |
| 12 | J04080 | C1S | complement component 1, s subcomponent | 64 |
| 12 | AA593793 | HEBP | heme-binding protein | 65 |
| 12 | U31525 | GYG | glycogenin | 66 |
| 12 | M63959 | LRPAP1 | low density lipoprotein-related protein- | 67 |
| | | | associated protein 1 (alpha-2-macroglobulin | |
| | | | receptor-associated protein 1) | |
| 12 | X04701 | C1R | complement component 1, r subcomponent | 68 |
| 12 | U44772 | PPT1 | palmitoyl-protein thioesterase 1 (ceroid- | 69 |
| | | | lipofuscinosis, neuronal 1, infantile) | |
| 12 | N27409 | RPS23 | ribosomal protein S23 | 70 |
| 11 | M29877 | FUCA1 | fucosidase, alpha-L-1, tissue | 71 |
| 11 | K03000 | ALDH1 | aldehyde dehydrogenase 1, soluble | 72 |
| 11 | AI061385 | SC5DL | sterol-C5-desaturase (fungal ERG3, delta-5- | 73 |
| | | | desaturase)-like | |
| 11 | X04481 | C2 | complement component 2 | 74 |
| 11 | AA682870 | CCND2 | cyclin D2 | 75 |
| 11 | AF055066 | HLA-F | major histocompatibility complex, class I, F | 76 |
| 11 | AU155489 | MMP7 | matrix metalloproteinase 7 (matrilysin, | 77 |
| | | | uterine) | |
| 10 | D78014 | DPYSL3 | dihydropyrimidinase-like 3 | 78 |
| 10 | AA704399 | UBE2N | ubiquitin-conjugating enzyme E2N | 79 |
| | | | (homologous to yeast UBC13) | |
| 10 | D55654 | MDH1 | malate dehydrogenase 1, NAD (soluble) | 80 |
| 10 | AI985921 | CAV1 | caveolin 1, caveolae protein, 22 kD | 81 |
| 10 | AA809819 | CREG | cellular repressor of E1A-stimulated genes | 82 |
| 10 | AA777014 | DAB2 | disabled (Drosophila) homolog 2 (mitogen- | 83 |
| | | | responsive phosphoprotein) | |
| 10 | U07231 | GRSF1 | G-rich RNA sequence binding factor 1 | 84 |
| 10 | L13210 | LGALS3BP | lectin, galactoside-binding, soluble, 3 | 85 |
| | | | binding protein (galectin 6 binding protein) | |
| 10 | D87258 | PRSS11 | protease, serine, 11 (IGF binding) | 86 |
| 10 | AA432312 | TSPYL | TSPY-like | 87 |

ESTs and genes with unknown function

| | | | | |
|---|---|---|---|---|
| 12 | BF593563 | DKFZP564A | DKFZP564A2416 protein | 88 |
| | | 2416 | | |
| 12 | AI142828 | | Homo sapiens adlican mRNA, complete cds | 89 |
| 12 | AI185130 | KIAA0193 | KIAA0193 gene product | 90 |
| 11 | N70341 | KIAA0672 | ESTs | 91 |
| 11 | AA665097 | LOC51323 | hypothetical protein | 92 |
| 10 | D87465 | KIAA0275 | KIAA0275 gene product | 93 |
| 10 | Z25391 | KIAA0728 | KIAA0728 protein | 94 |
| 10 | AA447864 | KIAA1055 | KIAA1055 protein | 95 |
| 10 | AI343963 | PP2135 | PP2135 protein | 96 |
| 10 | AI366597 | | ESTs | 97 |

Genes with normalized expression ratios (cyst/normal) of ≧2.0 in more than 70% of the 14 case were examined. The left-most column indicates the numbers of samples in that category. Accession numbers, gene symbols and names were retrieved from the Unigene Database (build#131).

TABLE 4
Genes down-regulated in endometrial cysts throughout the menstrual cycle

| | Genbank | | | |
|---|---|---|---|---|
| | Accession | | | OEX |
| ratio ≦ 0.3 | No. | Abbreviation | Gene name | Assignment |
| 21 | D86724 | ARG2 | arginase, type II | 98 |
| 21 | D87116 | MAP2K3 | mitogen-activated protein kinase kinase 3 | 99 |
| 20 | AI339572 | KLF5 | Kruppel-like factor 5 (intestinal) | 100 |
| 20 | U19906 | AVPR1A | arginine vasopressin receptor 1A | 101 |
| 20 | Y10032 | SGK | serum/glucocorticoid regulated kinase | 102 |
| 20 | M68891 | GATA2 | GATA-binding protein 2 | 103 |
| 19 | AA121949 | BAG3 | BCL2-associated athanogene 3 | 104 |
| 19 | U83981 | GADD34 | growth arrest and DNA-damage-inducible 34 | 105 |
| 19 | X85133 | RBBP6 | retinoblastoma-binding protein 6 | 106 |
| 19 | D86956 | HSP105B | heat shock 105 kD | 107 |
| 19 | W20076 | NXF1 | nuclear RNA export factor 1 | 108 |
| 19 | AI367368 | FACL5 | long-chain fatty acid coenzyme A ligase 5 | 109 |
| 18 | L17131 | HMGIY | high-mobility group (nonhistone | 110 |
| | | | chromosomal) protein isoforms I and Y | |
| 18 | U09550 | OVGP1 | oviductal glycoprotein 1, 120 kD (mucin 9, | 111 |
| | | | oviductin) | |
| 18 | L16876 | CYP2C18 | cytochrome P-450 2C18 | 112 |
| 18 | U73843 | ELF3 | E74-like factor 3 (ets domain transcription | 113 |
| | | | factor, epithelial-specific) | |
| 18 | AI014398 | RASD1 | RAS, dexamethason-induced 1 | 114 |
| 17 | AA436509 | IER5 | Immediate early response 5 | 115 |
| 17 | X15729 | DDX5 | DEAD/H (Asp-Glu-Ala-Asp/His) box | 116 |
| | | | polypeptide 5 (RNA helicase, 68 kD) | |
| 17 | D13388 | HSJ2 | heat shock protein, DNAJ-like 2 | 117 |
| 17 | D90070 | PMAIP1 | phorbol2-myristate3-acetate-induced protein1 | 118 |
| 17 | D49547 | DNAJB1 | DnaJ (Hsp40) homolog, subfamily B, | 119 |
| | | | member 1 | |
| 17 | M16441 | LTA | lymphotoxin alpha (TNF superfamily, | 120 |
| | | | member 1) | |
| 17 | U91618 | NTS | neurotensin | 121 |
| 17 | L13943 | GK | glycerol kinase | 122 |
| 17 | U63329 | MUTYH | mutY () homolog | 123 |
| 17 | U40462 | ZNFN1A1 | zinc finger protein, subfamily 1A, 1 (Ikaros) | 124 |
| 17 | AA234506 | LRRFIP1 | leucine rich repeat (in FLII) interacting | 125 |
| | | | protein 1 | |
| 17 | AI249000 | LIM | LIM protein (similar to rat protein kinase C- | 126 |
| | | | binding enigma) | |
| 17 | AA496218 | STAG2 | stromal antigen 2 | 127 |

Genes with normalized expression ratios (cyst/normal) of ≦0.3 in more than 70% of the 23 cases were examined. The left-most column indicates the numbers of samples in that category. Accession numbers, gene symbols and names were retrieved from the Unigene Database (build#131).

TABLE 5
Genes down-regulated in endometrial cysts only during
the proliferative phase of the menstrual cycle

| | GenBank | | | |
|---|---|---|---|---|
| | Accession | | | OEX |
| ratio ≦ 0.3 | No. | Abbreviation | Gene name | Assignment |
| 9 | D86955 | SCYA20 | small inducible cytokine subfamily A (Cys— | 128 |
| | | | Cys), member 20 | |
| 9 | D87953 | NDRG1 | N-myc downstream regulated | 129 |
| 8 | AF037335 | CA12 | carbonic anhydrase XII | 130 |
| 8 | D37766 | LAMB3 | laminin, beta 3 (nicein (125 kD), kalinin | 131 |
| | | | (140 kD), BM600 (125 kD)) | |
| 8 | AA053789 | ZNF216 | zinc finger protein 216 | 132 |
| 8 | U62015 | CYR61 | cysteine-rich, angiogenic inducer, 61 | 133 |
| 8 | U67784 | RDC1 | G protein-coupled receptor | 134 |
| 7 | AI081684 | VNN1 | vanin 1 | 135 |
| 7 | X97324 | ADFP | adipose differentiation-related protein | 136 |
| 7 | AA634090 | HNRPA1 | heterogeneous nuclear ribonucleoprotein A1 | 137 |
| 7 | U25997 | STC1 | stanniocalcin 1 | 138 |
| 7 | AA977557 | GOLPH2 | golgi membrane protein GP73 | 139 |
| 7 | Z46629 | SOX9 | SRY (sex determining region Y)-box 9 | 140 |
| | | | (campomelic dysplasia, autosomal sex- | |
| | | | reversal) | |
| 7 | AB001636 | DDX15 | DEAD/H (Asp-Glu-Ala-Asp/His) box | 141 |
| | | | polypeptide 15 | |

Genes with normalized expression ratios (cyst/normal) of ≦0.3 in more than 70% of the nine cases were examined. The left-most column indicates the numbers of samples in that category. Accession numbers, gene symbols and names were retrieved from the Unigene Database (build#131).

TABLE 6
Genes down-regulated in endometrial cysts only during
the secretory phase of the menstrual cycle

| | GenBank | | | |
|---|---|---|---|---|
| | Accession | | | OEX |
| ratio ≦ 0.3 | No. | Abbreviation | Gene name | Assignment |
| 14 | AI298111 | MRPS2 | mitochondrial ribosomal protein S2 | 142 |
| 14 | AA534943 | SCYB14 | small inducible cytokine subfamily B (Cys- | 143 |
| | | | X-Cys), member 14 (BRAK) | |
| 13 | X58295 | GPX3 | glutathione peroxidase 3 (plasma) | 144 |
| 13 | AA565113 | DRPLA | dentatorubral-pallidoluysian atrophy | 145 |
| | | | (atrophin) | |
| 12 | T84015 | PLEC1 | plectin 1, intermediate filament binding | 146 |
| | | | protein, 500 kD | |
| 12 | AA279817 | GADD45B | growth arrest and DNA-damage-inducible, | 147 |
| | | | beta | |
| 12 | AI160184 | LOC51673 | brain specific protein | 148 |
| 12 | AW162122 | APC4 | anaphase-promoting complex subunit 4 | 149 |
| 12 | X03438 | CSF3 | colony stimulating factor 3 (granulocyte) | 150 |
| 12 | AF007162 | CRYAB | crystallin, alpha B | 151 |
| 12 | M60974 | GADD45A | growth arrest and DNA-damage-inducible, | 152 |
| | | | alpha | |
| 12 | X03473 | H1F0 | H1 histone family, member 0 | 153 |
| 12 | M69226 | MAOA | monoamine oxidase A | 154 |
| 12 | M95548 | SLC3A1 | solute carrier family 3 (cystine, dibasic and | 155 |
| | | | neutral amino acid transporters, activator of | |
| | | | cystine, dibasic and neutral amino acid | |
| | | | transport), member 1 | |
| 12 | AI349114 | TCTE1L | t-complex-associated-testis-expressed 1 -like | 156 |
| 11 | AA639795 | FRSB | phenylalanyl-tRNA synthetase beta-subunit | 157 |
| 11 | AF073710 | RGS9 | regulator of G-protein signalling 9 | 158 |
| 11 | AF039691 | HDAC5 | histone deacetylase 5 | 159 |
| 11 | L34059 | CDH4 | cadherin 4, type 1, R-cadherin (retinal) | 160 |
| 11 | D10922 | FPRL1 | formyl peptide receptor-like 1 | 161 |
| 11 | Y10313 | IFRD1 | interferon-related developmental regulator 1 | 162 |
| 11 | AI186556 | PISD | phosphatidylserine decarboxylase | 163 |
| 11 | L26260 | STK19 | serine/threonine kinase 19 | 164 |
| 11 | AA419482 | LOC54518 | similar to proline-rich protein 48 | 165 |
| 11 | L01100 | ICA1 | islet cell autoantigen 1 (69 kD) | 166 |
| 11 | AF014398 | IMPA2 | inositol(myo)(or 4)-monophosphatase 2 | 167 |
| 11 | AA676322 | MTF1 | metal-regulatory transcription factor 1 | 168 |
| 10 | AI126155 | CUL3 | cullin 3 | 169 |
| 10 | M11717 | HSPA1A | heat shock 70 kD protein 1A | 170 |
| 10 | U08015 | NFATC1 | nuclear factor of activated T-cells, | 171 |
| | | | cytoplasmic, calcineurin-dependent 1 | |
| 10 | AA608780 | GKP2 | Glycerol kinase pseudogene 2 | 172 |
| 10 | D16581 | NUDT1 | nudix (nucleoside diphosphate linked moiety | 173 |
| | | | X)-type motif 1 | |
| 10 | AF066859 | PYGM | phosphorylase, glycogen; muscle (McArdle | 174 |
| | | | syndrome, glycogen storage disease type V) | |
| 10 | AA312113 | RBL1 | retinoblastoma-like 1 (p107) | 175 |
| 10 | AA912674 | JAM2 | vascular endothelial junction-associated | 176 |
| | | | molecule | |
| 10 | AI261581 | AGPS | alkylglycerone phosphate synthase | 177 |
| 10 | M78798 | CERD4 | Cer-d4 (mouse) homolog | 178 |
| 10 | M31452 | C4BPA | complement component 4-binding protein, | 179 |
| | | | alpha | |
| 10 | U48734 | ACTN4 | actinin, alpha 4 | 180 |
| 10 | M16451 | CKB | creatine kinase, brain | 181 |
| 10 | M63582 | TRH | thyrotropin-releasing hormone | 182 |
| 10 | X16940 | ACTG2 | actin, gamma 2, smooth muscle, enteric | 183 |
| 10 | AI278397 | DLX5 | distal-less homeo box 5 | 184 |
| 10 | N70019 | MT1E | metallothionein 1E (functional) | 185 |

Genes with normalized expression ratios (cyst/normal) of ≦0.3 in more than 70% of the 14 cases were examined. The left-most column indicates the numbers of samples in that category. Accession numbers, gene symbols and names were retrieved from the Unigene Database (build#131).

TABLE 7
Genes up-regulated in endometrial cysts throughout the menstrual cycle

| Accession | | | OEX |
|---|---|---|---|
| No. | Symbol | Gene name | Assignment |
| M63262 | ALOX5AP | arachidonate 5-lipoxygenase-activating | 1 |
| | | protein | |
| X63629 | CDH3 | cadherin 3, type 1, P-cadherin (placental) | 6 |
| Z22970 | CD163 | CD 163 antigen | 186 |
| X04701 | C1R | complement component 1, r subcomponent | 68 |
| M20431 | | DC classII histocompatibility antigen alpha-chain | 187 |
| X00637 | HP | haptoglobin | 9 |
| K03431 | HPR | haptoglobin-related protein | 188 |
| V00497 | HBB | hemoglobin, beta | 189 |
| U01317 | HBD | hemoglobin, delta | 190 |
| AA583491 | HCA112 | hepatocellular carcinoma-associated antigen 112 | 2 |
| X00457 | | Human mRNA for SB classII | 3 |
| | | histocompatibility antigen alpha-chain | |
| M87789 | IGHG3 | immunoglobulin heavy constant gamma 3 (G3m marker) | 191 |
| M87790 | IGLλ | immunoglobulin lambda locus | 192 |
| K01171 | HLA-DRA | major histocompatibility complex, class II, DR alpha | 8 |
| X07819 | MMP7 | matrix metalloproteinase 7 (matrilysin, uterine) | 193 |
| U44403 | SLA | Src-like-adapter | 28 |

Other Genes

| | | |
|---|---|---|
| AI142828 | Homo sapiens adlican mRNA, complete cds | 89 |
| K01505 | ESTs | 5 |
| AI138545 | ESTs | 194 |
| AI310156 | ESTs, Weakly similar to A4P_HUMAN | 15 |
| | INTESTINAL MEMBRANE A4 | |
| | PROTEIN [H. sapiens] | |

Genes with normalized expression ratios (cyst/normal) of ≧5.0 in more than 50% of the 23 cases examined were selected. Accession Nos., gene symbols and names were retrieved from the Unigene Database (build #131).

TABLE 8
Genes up-regulated in endometrial cysts from patients
in the proliferative phase of the menstrual cycle

| Accession | | | OEX |
|---|---|---|---|
| No. | Symbol | Gene name | Assignment |
| U07919 | ALDH6 | aldehyde dehydrogenase 6 | 195 |
| M86511 | CD14 | CD14 antigen | 11 |
| W67577 | CD74 | CD74 antigen (invariant polypeptide of major | 196 |
| | | histocompatibility complex, class II antigen- | |
| | | associated) | |
| U58514 | CHI3L2 | chitinase 3-like 2 | 197 |
| X02761 | FN1 | fibronectin 1 | 198 |
| AA854147 | HDAC7A | Histone deacetylase 7A | 199 |
| M83202 | LTF | lactotransferrin | 21 |
| X83006 | LCN2 | lipocalin 2 (oncogene 24p3) | 200 |
| M33906 | HLA- | major histocompatibility complex, class II, | 201 |
| | DQA1 | DQ alpha 1 | |
| M15800 | MAL | mal, T-cell differentiation protein | 202 |
| AF034374 | | molybdenum cofactor biosynthesis protein A; | 20 |
| | | molybdenum cofactor biosynthesis protein C | |
| H48536 | PCAF | p300/CBP-associated factor | 203 |
| U02632 | KCNMA1 | potassium large conductance calcium- | 204 |
| | | activated channel, subfamily M, alpha | |
| | | member 1 | |
| AA972852 | RBP1 | retinol-binding protein 1, cellular | 205 |
| X00129 | RBP4 | retinol-binding protein 4, interstitial | 206 |
| L20688 | ARHGDIB | Rho GDP dissociation inhibitor (GDI) beta | 207 |
| AA778308 | RNASE1 | ribonuclease, RNase A family, 1 (pancreatic) | 17 |
| X51441 | SAA1 | serum amyloid A1 | 208 |
| M77349 | TGFBI | transforming growth factor, beta-induced, | 4 |
| | | 68 kD | |

Other Genes

| | | | |
|---|---|---|---|
| AA455877 | | Homo sapiens cDNA FLJ11177 fis, clone | 209 |
| | | PLACE1007402 | |
| AA772709 | | Homo sapiens cDNA FLJ13522 fis, clone | 210 |
| | | PLACE1005884 | |
| AA652120 | | Homo sapiens cDNA: FLJ21869 fis, clone | 211 |
| | | HEP02442 | |
| W87690 | | Homo sapiens cDNA: FLJ23173 fis, clone | 212 |
| | | LNG10019 | |
| AA938345 | | Homo sapiens mRNA; cDNA | 213 |
| | | DKFZp564N1116 (from clone | |
| | | DKFZp564N1116) | |
| AA577682 | | Homo sapiens mRNA; cDNA | 214 |
| | | DKFZp586F1822 (from clone | |
| | | DKFZp586F1822) | |
| N36090 | FLJ10895 | hypothetical protein FLJ10895 | 215 |
| AA164951 | | EST | 216 |
| H12942 | | ESTs | 47 |
| AA179600 | | ESTs | 217 |
| AI281932 | | ESTs | 218 |
| AI130715 | | ESTs | 219 |
| N66074 | | EST | 220 |
| AI038441 | | ESTs | 48 |
| W04197 | | ESTs | 221 |
| AA992745 | | ESTs | 222 |
| BE894625 | | EST | 53 |
| AA994249 | | ESTs | 49 |
| AA921763 | | ESTs | 52 |

Genes with normalized expression ratios (cyst/normal) of ≧5.0 in more than 50% of the nine cases examined were selected. Accession Nos., gene symbols and names were retrieved from the Unigene Database (build #131).

TABLE 9
Genes up-regulated in endometrial cysts during
the secretory phase of the menstrual cycle

| Accession | | | |
|---|---|---|---|
| No. | Symbol | Gene name | |
| K03000 | ALDH1 | aldehyde dehydrogenase 1, soluble | 72 |
| S67310 | BF | B-factor, properdin | 62 |
| X56667 | CALB2 | calbindin 2, (29 kD, calretinin) | 223 |
| T93566 | CPE | carboxypeptidase E | 224 |
| AA319695 | CEBPD | CCAAT/enhancer binding protein (C/EBP), delta | 60 |
| M14354 | F13A1 | coagulation factor XIII, A1 polypeptide | 225 |
| J04080 | C1S | complement component 1, s subcomponent | 64 |
| X04481 | C2 | complement component 2 | 74 |
| W45244 | C3 | complement component 3 | 58 |
| AA682870 | CCND2 | cyclin D2 | 75 |
| AF019413 | CYP21A2 | cytochrome P450, subfamily XXIA (steroid | 226 |
| | | 21-hydroxylase, congenital adrenal | |
| | | hyperplasia), polypeptide 2 | |
| D78014 | DPYSL3 | dihydropyrimidinase-like 3 | 78 |
| AA313118 | DUSP10 | dual specificity phosphatase 10 | 227 |
| AA573809 | ITLN | Intelectin | 228 |
| J00269 | KRT6A | keratin 6A | 229 |
| M81141 | HLA- | major histocompatibility complex, class II, DQ | 10 |
| | DQB1 | beta 1 | |
| D28124 | NBL1 | neuroblastoma, suppression of tumorigenicity 1 | 59 |
| U20157 | PLA2G7 | phospholipase A2, group VII (platelet- | 230 |
| | | activating factor acetylhydrolase, plasma) | |
| D87258 | PRSS11 | protease, serine, 11 (IGF binding) | 86 |
| L35545 | PROCR | protein C receptor, endothelial (EPCR) | 231 |
| U70136 | PRG4 | proteoglycan 4, (megakaryocyte stimulating | 13 |
| | | factor, articular superficial zone protein) | |
| AA263000 | RNASE6 | ribonuclease, RNase A family, k6 | 232 |
| AF026692 | SFRP4 | secreted frizzled-related protein 4 | 233 |
| D29992 | TFPI2 | tissue factor pathway inhibitor 2 | 234 |
| X51630 | WT1 | Wilms tumor 1 | 235 |

Other Genes

| | | | |
|---|---|---|---|
| AA429149 | C11ORF9 | chromosome 11 open reading frame 9 | 236 |
| AI366242 | | ESTs | 237 |
| D85376 | | ESTs | 7 |
| M32093 | | ESTs | 14 |
| AA424195 | | ESTs | 238 |
| AI224952 | | ESTs | 239 |
| AA669034 | | Homo sapiens cDNA: FLJ23125 fis, clone LNG08217 | 240 |
| L02326 | | Homo sapiens clone Hu lambda7 lambda-like | 241 |
| | | protein (IGLL2) gene, partial cds | |
| D87465 | KIAA0275 | KIAA0275 gene product | 93 |
| AA528009 | KIAA1077 | KIAA1077 protein | 242 |

Genes with normalized expression ratios (cyst/normal) of ≧5.0 in more than 50% of the 14 cases examined were selected. Accession Nos., gene symbols and names were retrieved from the Unigene Database (build #131).

Diagnosing Ovarian Endometriosis

The present invention provides a method for diagnosing ovarian endometriosis. According to the present method, a subject suffering from or in the risk of developing ovarian endometriosis can be diagnosed. The subject to be diagnosed according to the present method is preferably a mammal including human, non-human primates (monkey, baboon, chimpanzee, etc.), mouse, rat, guinea-pig, rabbit, dog, cat, sheep, pig, horse and cow.

Ovarian endometriosis may be diagnosed by examining the expression of one or more OEX-associated genes or proteins in a subject-derived biological sample. Specifically, the method of diagnosing ovarian endometriosis of the present invention involves determining (measuring) the expression level of at least one and up to all the OEX associated genes listed in Tables 1-9 or their proteins. Using sequence information provided by the GenBank database entries for the known sequences, the ovarian endometriosis-associated genes are detected and measured using techniques well known to one of ordinary skill in the art. The expression of 1, 2, 3, 4, 5, 25, 35, 50, 100 or more of OEX 1-242 or their proteins may be determined and if desired, expression of these nucleotides or proteins can be determined along with other nucleotides or proteins whose expression level is known to be altered according to conditions, e.g., ovarian endometriosis or non-ovarian endometriosis.

According to the present method, the expression level of one or more of the OEX associated genes or proteins in a biological sample derived from a subject is compared to a control level of the same genes or proteins. The subject-derived biological sample may be any sample so long as the expression of one or more OEX associated genes or proteins can be detected in a patient of ovarian endometriosis and includes, but is not limited to, blood, serum and tissue samples obtained from a test subject. Blood and serum samples can be readily obtained from peripheral blood vessels. Tissue samples include individual cells and cell populations such as test cells obtained from a bodily tissue or a bodily fluid (biological fluid such as blood, serum and urine). Preferably, the tissue samples comprise a test cell population comprising an epithelial cell derived from a tissue that is known to be or suspected to be an endometrial cyst.

The phrase “control level” refers to the expression level of the OEX associated genes or proteins in a reference sample. The reference sample should be the same type of sample as the biological sample examined in the present method of diagnosis. Namely, when the subject-derived biological sample is blood, then blood is used as a reference sample to determine the control level. The reference sample is derived from a subject or a population of subjects for whom the parameter to be compared is known, i.e., endometriotic or non-endometriotic. Alternatively, the control level may be calculated from a database of molecular information derived from subject(s) for which the assay parameter or condition is known.

According to the present method of diagnosing ovarian endometriosis, the expression level of the OEX associated genes or proteins in a subject-derived biological sample may be compared to multiple control levels of the nucleotide(s) or protein(s). The control levels may be derived from biological samples with different known parameters (i.e., endometriotic or non-endometriotic). Thus, the expression level of the genes or proteins in a subject-derived biological sample may be compared to the control level corresponding to ovarian endometriosis patient (ovarian endometriosis control level), and then to the control level corresponding to non-ovarian endometriosis subject (normal control level). Thus, the control level may be a single expression pattern derived from a single reference population or may be a plurality of expression patterns. For example, the control level can be a database of expression patterns derived from previously tested samples.

Whether an expression level in a subject-derived biological sample as compared to that in a control level indicates ovarian endometriosis or a predisposition thereto depends on the kind of the sample used for determining the control level. For example, when the control level is detected in a reference sample derived from a non-endometriotic individual, similar expression levels between the subject-derived biological sample and the reference sample indicates that the subject is non-endometriotic. Herein, such control levels are referred to as “normal control levels”. A normal control level indicates an expression level detected in a normal, healthy individual or in a population of individuals known not to be suffering from ovarian endometriosis. A normal healthy individual is one with no clinical symptoms of ovarian endometriosis. Conversely, when the control level is detected in a reference sample derived from an endometriotic patient, a similar expression profile between the subject-derived biological sample and the reference sample indicates that the subject suffers from ovarian endometriosis. Herein, such control levels are referred to as “ovarian endometriosis control levels” or “disease control levels”. The phrase “ovarian endometriosis (or disease) control level” refers to the expression profile of the OEX associated genes or proteins found in a patient or a population of patients suffering from endometriosis.

The expression level of an OEX associated gene or protein in a subject-derived biological sample is considered to be altered when the sample expression level differs from a control level by more than 0.1, 0.5, 1.0, 2.0, 5.0, 10.0 or more folds. Alternatively, an expression level in a subject-derived biological sample increased or decreased by 1%, 5%, 10%, 25%, 50% or more as compared to a control level indicates alteration of the expression level in the subject sample.

An increase in the expression level of the OEX 1-97 and 186-242 or their proteins detected in a subject-derived biological sample as compared to a normal control level indicates that the subject suffers from or is at a risk of developing ovarian endometriosis. In contrast, a decrease in the expression level of the OEX 98-185 or their proteins detected in a subject-derived biological sample as compared to a normal control level indicates that the subject suffers from or is at a risk of developing ovarian endometriosis.

A decrease or similarity in the expression level of OEX 1-97 and 186-242 detected in a subject-derived biological sample compared to an ovarian endometriosis control level indicates that the subject suffers from or is at risk of developing ovarian endometriosis. In contrast, an increase or similarity in the level of OEX 98-185 detected in a subject-derived biological sample compared to a normal control level indicates that said subject suffers from or is at risk of developing ovarian endometriosis. Alteration in the expression of one or more of the ovarian endometriosis-associated genes or proteins in a subject-derived biological sample as compared to a normal control level indicates that the subject suffers from or is at risk of developing ovarian endometriosis. To obtain a more reliable diagnosis result, it is preferable to examine the expression level of multiple OEX nucleotides. If the expression level of 1%, 5%, 25%, 50%, 60%, 80%, 90% or more of the OEX 1-242 is altered in a subject-derived biological sample, the probability that the subject suffers from or is at risk of developing ovarian endometriosis becomes quite high.

When required, comparison of the expression level in a subject-derived biological sample to a control level can be conducted with respect to a control nucleic acid or protein whose expression is independent of the parameter or condition being measured. A “control nucleic acid or protein” is one whose expression level is known not to differ between the endometriotic or non-endometriotic state of the individual to be tested. Expression levels of a control nucleic acid or protein in the test and reference samples can be used to normalize the levels in the compared populations. A control nucleic acid is also called “housekeeping gene” and genes such as β-actin, glyceraldehyde 3-phosphate dehydrogenase or ribosomal protein P1 may be used in the present invention.

Expression of the genes disclosed herein may be determined at the RNA level using any method known in the art. For example, sequences within the sequence database entries corresponding to OEX nucleic acid sequences can be used to construct probes for detecting OEX RNAs by, e.g., Northern blot hybridization analyses. A probe preferably includes at least 10, 20, 50, 100, 200 or more continuous nucleotides of a reference sequence (i.e., the nucleotide sequence of OEX nucleic acids (Tables 1-9)). Alternatively, the expression level is measured using reverse-transcription-based PCR (RT-PCR) assays, e.g., using primers specific for the OEX nucleic acid sequences.

When alterations in gene expression are associated with gene amplification or deletion, the gene (DNA sequence) in a subject-derived biological sample may be compared to that of a reference cell population to determine whether the subject suffers from or is at risk of developing ovarian endometriosis.

According to the method of the present invention, protein expression levels can be determined by measuring the levels or the biological activity of the expressed polypeptides encoded by the genes described herein. The biological activities of respective polypeptides encoded by the OEX-associated genes are also well known in the art, and one skilled in the art can adopt appropriate conventional methods for measuring the biological activities of the polypeptides depending on the kind of polypeptide to be measured. Such methods include, but not limited to, immunoassays using antibodies against polypeptides encoded by the genes.

In the context of the instant invention, suitable immunoassays for measuring the expression level of a polypeptide include all kinds of known immunoassays, so long as it can be used for the detection of OEX polypeptides in a subject-derived biological sample, and include, but are not limited to, competitive assays and non-competitive assays; homogeneous assays and heterogeneous assays; homologous assays, heterologous assays and hetero-antibody assays; and assays wherein labeling substances, such as radioisotopes, enzymes, lanthanides, fluorescent substances, luminescent substances, free radicals and the like, are used. Competitive assays involve the competitive binding of labeled and non-labeled substances to a small amount of antibody. Exemplary competitive assays include, but are not limited to, radioimmunoassays (RIA) wherein the antigen is labeled with radioisotope; enzyme immunoassays (EIA) wherein enzymes are used as the labeling substance; time-resolved fluoroimmunoassays (TR-FIA), including, for example, dissociation-enhanced lanthanide flurorometric immunoassays (DELFIA®) wherein lanthanides are used as the labeling substance; fluoroimmunoassays (FIA) using fluorescent substances that emit fluorescent upon irradiation with ultraviolet; and luminescence immunoassays (LIA) using chemical luminescent substances. In non-competitive assays, sandwich binding is performed wherein the target substance is exposed to excess antibody and then detected with labeled antibodies. Similarly to the competitive assays, the non-competitive assays can be also conducted using radioisotopes, enzymes, lanthanides, fluorescent substances, luminescent substances and such, and include, but are not limited to, immunoradiometric assays (IRMA), enzyme-linked immunosorbent assays (ELISA), immunofluorometric assays (IFMA), time-resolved immunofluorometric assays (TR-IFMA), immunoluminometric assays (ILMA), and the like. In homogenous assays, measurement is conducted in a solution state, whereas in heterogeneous assays involve immobilizing the antibody on a solid matrix. The term “homologous assay” refers to an assay system wherein the target to be detected, standard sample and antibodies are all derived from the same species. Conversely, heterologous assays utilize an assay system for one animal species for the detection of antigens in an animal of another species (e.g., bovine against goat, rat against mouse, etc.). When the heterologous assay does not meet a good result, the use of homologous standard samples and heterologous antibodies are sometimes successful in RIA and such, which method is classified as a hetero-antibody assay.

A preferable immunoassay method for the present invention is ELISA. According to a preferred embodiment of the method of the present invention, the expression level of the protein is determined by ELISA using a polyclonal antibody against an OEX polypeptide as the labeled antibody, and a monoclonal antibody against the protein as the immobilized antibody. A small amount of the OEX polypeptide in a subject-derived biological sample is expected to be efficiently detected by such a method. The present invention refers to the use of antibodies against OEX polypeptides or a fragment of such an antibody. As used herein, the term “antibody” refers to an immunoglobulin molecule having a specific structure that interacts (binds) specifically with a molecule comprising the antigen used for synthesizing the antibody (i.e., OEX polypeptides or fragments thereof) or with an antigen closely related to it. An antibody that binds to an OEX polypeptide may be in any form, such as monoclonal or polyclonal antibodies, and includes antiserum obtained by immunizing an animal, such as a rabbit, with the polypeptide, all classes of polyclonal and monoclonal antibodies, human antibodies and humanized antibodies produced by genetic recombination.

Furthermore, in the context of the present invention, the antibody may be a fragment of an antibody or a modified antibody, so long as it binds to its antigen (i.e., OEX polypeptides) and such binding can be detected. For instance, the antibody fragment may be Fab, F(ab′)2, Fv or single chain Fv (scFv), in which Fv fragments from H and L chains are ligated by an appropriate linker (Huston et al. (1988) Proc. Natl. Acad. Sci. USA 85: 5879-83). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co et al. (1994) J. Immunol. 152: 2968-76; Better M. and Horwitz (1989) Methods Enzyme. 178:476-96; Pluckthun and Skerra (1989) Methods Enzyme. 178: 497-515; Lamoyi (1986) Methods Enzyme. 121: 652-63; Rousseaux et al. (1986) Methods Enzyme. 121:663-9; Bird and Walker (1991) Trends Biotechnol. 9: 132-7).

An antibody may be modified by conjugation with a variety of molecules including, but not limited to, matrices for immobilizing the protein and labels for detecting the antibody. Alternatively, the modified antibody can be obtained through chemical modification of the antibody. Such modification methods are conventional in the field.

An antibody may be directly or indirectly labeled with labeling substances, such as radioisotopes (³H, ¹⁴C, ³²P, ³³P, ³⁵S, ¹²⁵I, ¹³¹I, etc.), enzymes (alkaline phosphatase, peroxidase, β-glucuronidase, β-galactosidase, β-glucosidase, glucose oxidase, glucose-6-phosphate dehydrogenase, alcohol dehydrogenase, penicillinase, catalase, urease, luciferase, etc.), fluorescent substances (fluorescein isothiosyanete (FITC), rhodamine, etc.), lanthanides (europium (EU), etc.), luminescent substances (luminol, acridinium ester, etc.), free radicals (piperidine-N-oxide derivatives, pyrrolidine-N-oxide derivatives, oxazolidine-N-oxide derivatives, etc.) and biotin/avidin, by conventional methods. Antibodies may be directly labeled with the above labeling substances through the use of a cross-linker (e.g., N,N′-orthophenylene dimaleimide, 4,4′-dithiopyridine, etc.). Indirect labeling methods of antibodies include conjugating of the antibodies with small molecular weight haptens, such as biotin, dinitrophenyl and pyridoxal, wherein detection involves a substance that binds to the hapten(s).

When the antibody is labeled with a radioisotope, the detection or measurement can be carried out by liquid scintillation. Alternatively, antibodies labeled with enzymes can be detected or measured by adding a substrate of the enzyme to detect the enzymatic change of the substrate, such as generation of color, with absorptiometer. Alternatively, enzymes such as peroxidase may be used for the present method, and the peroxidase can be detected by reacting o-phenylenediamine under the existence of hydrogen peroxide and measuring the absorbance at a wave length of A490. Further, in cases where a fluorescent substance is used as the label, the bound protein may be detected or measured using a fluorophotometer. Furthermore, a polyclonal antibody against an OEX polypeptide for ELISA may be biotinylated using, for example, the ECL Protein Biotinylation Module (Amersham Biosciences). Following binding with proteins in a biological sample, the biotin conjugated on the polyclonal antibody is reacted with avidin. Methods for measuring the avidins bound to biotins are known in the art and include methods wherein the avidin is conjugated with detectable enzymes. When lanthanides, such as Eu, are used as the labeling substance, the fluorescence (615 nm) emitted upon excitation with a light of a wavelength of 340 nm can be detected. Kits using lanthanides are commercially available (e.g., those from PerkinElmer called Dissociation-enhanced lanthanide fluorometric immunoassay (DELFIA)). Free radical labels can be detected based on the change in the spectrum of its electron spin resonance (ESR).

Matrices to immobilize an antibody (polyclonal or monoclonal, though polyclonals are preferred for their highly efficient detection of antigens in a sample) are not restricted in any way and include, but are not limited to, matrices in the form of plates, beads, fibers, wells, etc. made of polystyrene, polyethyrene, polyvinyl chloride, polyester, nylon, polyacetal, fluoroplastic, glass, cellulose, agarose, metal, etc. The immobilization of an antibody to a matrix may be conducted according to any conventional chemical binding or physical adsorption methods. An antibody may be indirectly immobilized on a matrix using secondary antibodies recognizing antibodies (e.g., anti-IgG antibody), or protein A or protein G that binds to the constant region of an antibody.

Assessing Efficacy of Treatment of Ovarian Endometriosis in a Subject

The differentially expressed OEX nucleotides or polypeptides identified herein also allow for the course of treatment of ovarian endometriosis to be monitored. According to the method of the present invention, a biological sample, such as blood, serum, or tissue samples, is obtained from a subject undergoing treatment for ovarian endometriosis. The method for assessment can be conducted in a manner analogous to the method of diagnosing ovarian endometriosis of the present invention described above.

Specifically, when required, biological samples can be obtained from the subject at various time points before, during or after the treatment. The expression level of one or more of the OEX associated genes in the biological sample is then determined and compared to a control level, for example, a reference sample derived from an individual whose state of ovarian endometriosis (i.e., endometriotic or non-endometriotic) is known. The control level is determined in a biological sample that has not been exposed to the treatment of interest.

When the control level is derived from a biological sample taken from an individual not suffering from ovarian endometriosis (i.e., a normal control level), a similarity between the expression level in the subject-derived biological sample and the normal control level indicates that the treatment is efficacious. A difference between the expression level of the OEX nucleotides or OEX polypeptides in the subject-derived biological sample and the normal control level indicates a less favorable clinical outcome or prognosis.

Furthermore, over-expressed OEX protein expression levels determined in a subject-derived biological sample obtained after treatment (i.e., post-treatment levels) can be compared to the protein expression levels determined in a subject-derived biological sample obtained prior to treatment onset (i.e., pre-treatment levels). A decrease in expression levels of the protein in post-treatment samples also suggests that the treatment is efficacious.

As used herein, the term “efficacious” indicates that the treatment leads to a reduction in the expression of a pathologically up-regulated protein (OEX 1-97 and OEX 186-242), increase in the expression of a pathologically down-regulated gene (OEX 98-185) or a decrease in size, prevalence or proliferating potential of endometrial cysts in a subject. When a treatment is applied prophylactically, “efficacious” indicates that the treatment retards or prevents formation of ovarian endometrial cysts. The assessment of endometrial cysts can be made using standard clinical protocols.

The efficaciousness of a treatment is determined in association with any known method for diagnosing or treating ovarian endometriosis. Ovarian endometriosis is diagnosed for example, by identifying symptomatic anomalies, e.g., progressive dysmenorrhea, dyspareunia, chronic pelvic pain and infertility, along with surgical identification of endometrial glands and stroma outside the uterus.

Assessing the Prognosis of a Subject with Ovarian Endometriosis

The present invention further provides a method of assessing the prognosis of a subject with ovarian endometriosis by comparing the expression level of one or more OEX nucleotides or polypeptides in a subject -derived biological sample, such as blood or tissue samples, to a control level. Alternatively, the expression level of one or more OEX nucleotides or polypeptides in a biological sample derived from subjects may be measured over a spectrum of disease stages to assess the prognosis of the subject. The method for assessment can be conducted in a manner analogous to the method of diagnosing ovarian endometriosis of the present invention described above.

For example, a decrease in the expression level of one or more of OEX 98-185 or their gene products compared to a normal control level or an increase in the expression level of one or more of OEX 1-97 and 186-242 or their gene products in the subject-derived sample as compared to a normal control level indicates a less favorable prognosis for the subject. Conversely, an increase in the expression level of one or more of OEX 98-185 or their gene products indicates a more favorable prognosis, and a decrease in or similarity between the expression level of OEX 1-97 and 186-242 or their gene products in the subject-derived sample as compared to a normal control level indicates a more favorable prognosis for the subject.

Ovarian Endometriosis Reference Expression Profile

An ovarian endometriosis reference expression profile is provided by the present invention. Such expression profiles of the present invention comprise a pattern of gene expression of two or more OEX nucleotides (OEX 1-242) in a cell of endometrial cysts or a normal healthy endometrial cell (non-endometriotic cell). Furthermore, the present expression profile may comprise a pattern of gene expression of two or more genes of OEX 1-97 and OEX 186-242 or OEX 98-185. The expression profile can be used in diagnosing ovarian endometriosis or a predisposition to developing ovarian endometriosis in a subject, monitoring the course of treatment of ovarian endometriosis and assessing prognosis of a subject with ovarian endometriosis.

Screening Compounds that Alter Ovarian Endometriosis-Associated Gene Expression or the Biological Activity of a Polypeptide Encoded by the Ovarian Endometriosis-Associated Gene

The invention further provides methods of screening a compound that alters, i.e., inhibits or enhances the expression of a marker gene, ovarian endometriosis-associated gene (OEX 1-242), by (1) contacting a test compound with a test cell expressing an ovarian endometriosis-associated gene or a cell into which a vector comprising a reporter gene linked downstream of a transcriptional regulatory region of an ovarian endometriosis-associated gene has been introduced; (2) determining the expression level of the ovarian endometriosis-associated gene; and (3) selecting the compound that alters the expression level compared to that in the absence of the test compound. The method is based on screening a compound to determine if it converts an expression profile of OEX 1-242 characteristic of an ovarian endometriosis state to a pattern indicative of a non-ovarian endometriosis state.

A decrease in the expression level of an ovarian endometriosis-associated gene or a reporter gene linked downstream of a transcriptional regulatory region of an ovarian endometriosis-associated gene compared to the expression level detected in the absence of a test compound indicates that the test compound is an inhibitor. Alternatively, enhanced expression level of the genes compared to the expression level detected in the absence of a test compound indicates the test compound to function as an enhancer. The expression level detected in the absence of a test compound may be a normal control level or ovarian endometriosis control level.

If a gene up-regulated in the endothelial cells of endometrial cysts of patients with ovarian endometriosis (e.g., any one selected from OEX 1-97 and OEX 186-242) or the transcriptional regulatory region thereof is used in the screening method, a compound that inhibits the expression of the gene is expected to inhibit endometriosis.

Alternatively, if a gene down-regulated in the endothelial cells of endometrial cysts of patients with ovarian endometriosis (e.g., any one selected from OEX 98-185) or the transcriptional regulatory region thereof is used in the screening method, a compound that enhances the expression of the gene is expected to inhibit endometriosis.

In the method, a cell may be exposed to a test compound or a combination of test compounds (sequentially or consequentially). Compounds selected by the screening of the present invention serve as candidate compounds for treating or preventing ovarian endometriosis. Compounds effective in stimulating expression of under-expressed (down-regulated) marker genes or in suppressing expression of over-expressed (up-regulated) marker genes are deemed to lead to a clinical benefit. Therefore, such compounds are further tested for the ability to prevent endometrial cyst growth in endometrial glands and/or stroma of animals or test subjects. Moreover, compounds in which a part of the structure is converted by addition, deletion, substitution and/or insertion are also included in the compounds obtainable by the screening of the present invention.

The test cell used in the screening may be any cell so long as it expresses the ovarian endometriosis-associated gene. Furthermore, the test cell may be a test cell population consisting of multiple cells. For example, the test cell or test cell population contains an epithelial cell, such as those derived from an endometrial cyst. Moreover, the test cell or test cell population may be an immortalized cell or cell line derived from an endometrial cyst cell.

A transcriptional regulatory region of an ovarian endometriosis-associated gene can be obtained from genomic libraries using probes comprising the 5′ region of the OEX nucleotides (OEX 1-242). Any reporter gene may be used in the screening so long as its expression can be detected in the screening. Examples of reporter genes include the l-gal gene, CAT gene and luciferase gene. Detection of the expression of the reporter gene can be conducted based on conventional methods in accordance with the type of the reporter gene. Although there is no restriction on the cell into which the vector is introduced, preferable examples include epithelial cell.

The present invention further provides a method of screening for a compound that alters the activity of an ovarian endometriosis-associated gene. An embodiment of this screening method comprises the steps of: (a) contacting a test compound with a polypeptide encoded by an ovarian endometriosis-associated gene; (b) detecting the binding activity between the polypeptide and the test compound; and (c) selecting the compound that binds to the polypeptide.

In another embodiment of the method for screening a compound that alters the activity of an ovarian endometriosis-associated gene, the method utilizes the biological activity of an OEX polypeptide as an index. The screening method includes the steps of: (a) contacting a test compound with a polypeptide encoded by an ovarian endometriosis-associated gene; (b) detecting the biological activity of the polypeptide; and (c) selecting the compound that alters the biological activity of the polypeptide in comparison with the biological activity detected in the absence of the test compound.

The OEX polypeptides used for the present screening are selected from:
(1) a polypeptide comprising the amino acid sequence encoded by a polynucleotide selected from the group consisting of OEX 1-242;
(2) a polypeptide that comprises the amino acid sequence encoded by a polynucleotide selected from the group consisting of OEX 1-242, in which one or more amino acids are substituted, deleted and/or added and that has a biological activity equivalent to a protein consisting of the amino acid sequence encoded by the polynucleotide; and
(3) a polypeptide encoded by a polynucleotide that hybridizes under stringent conditions to a polynucleotide selected from the group consisting of OEX 1-242, wherein the polypeptide has a biological activity equivalent to a polypeptide consisting of the amino acid sequence encoded by the polynucleotide selected from the group consisting of OEX 1-242.

In the present invention, the phrase “biological activity” refers to activities such as occurrence, growth or proliferation of endometrial cyst cell. Whether an objective polypeptide has the biological activity or not can be judged by introducing the polypeptide or a DNA encoding the polypeptide into a cell, and detecting growth or proliferation of the cells, increase in colony forming activity, etc.

Methods for preparing polypeptides having the biological activity of a given protein are well known in the art and include methods introducing mutations into the protein. For example, one can prepare polypeptides having the biological activity of the OEX protein by introducing an appropriate mutation in the amino acid sequence of either of these proteins by site-directed mutagenesis (Hashimoto-Gotoh et al. (1995) Gene 152: 271-5; Zoller and Smith (1983) Methods Enzymol. 100: 468-500; Kramer et al. (1984) Nucleic Acids Res. 12: 9441-56; Kramer and Fritz (1987) Methods Enzymol. 154: 350-67; Kunkel (1985) Proc. Natl. Acad. Sci. USA 82: 488-92; Kunkel (1988) Methods Enzymol. 85: 2763-6). Amino acid mutations can occur in nature too. The OEX polypeptides include those having the amino acid sequences of the human OEX proteins in which one or more amino acids are mutated, provided the resulting mutated polypeptides have the biological activity of the OEX proteins. The number of amino acids to be mutated in such a mutant is generally 10 amino acids or less, preferably 6 amino acids or less and more preferably 3 amino acids or less.

An example of a polypeptide to which one ore more amino acid residues are added include fusion proteins containing the OEX protein. Fusion proteins can be made by techniques well known to those skilled in the art, such as linking DNA encoding the OEX protein with DNA encoding other peptides or proteins, so that the frames match, inserting the fused DNA into an expression vector and expressing it in a host. There is no restriction as to the peptides or proteins fused to the OEX protein and include FLAG (Hopp et al. (1988) Biotechnology 6: 1204-10), 6× His, 10× His, Influenza agglutinin, human c-myc fragment, VSP-GP fragment, p18HIV fragment, T7-tag, HSV-tag, E-tag, SV40T antigen fragment, Ick tag, α-tubulin fragment, B-tag, Protein C fragment, glutathione-S-transferase, immunoglobulin constant region, β-galactosidase, maltose binding protein, green fluorescence protein, etc. Vectors which can express a fusion protein with such peptides or proteins by the use of its multiple cloning sites are commercially available and can be used for obtaining fusion proteins to be used in the present screening.

An alternative method known in the art to isolate polypeptides having the biological activity of any of the OEX proteins is, for example, the method using hybridization technique (Sambrook et al. (1989) Molecular Cloning 2nd ed. 9.47-9.58, Cold Spring Harbor Lab. Press). One skilled in the art can readily isolate a DNA having high homology with a whole or part of the DNA sequence encoding an OEX protein, and isolate polypeptides having the biological activity of the OEX protein from the isolated DNA. The OEX polypeptides include those that are encoded by DNA that hybridize with a whole or part of a gene selected from OEX 1-242 and have the biological activity of the OEX protein. These polypeptides include mammal homologues corresponding to the protein derived from human (for example, a polypeptide encoded by a monkey, rat, rabbit and bovine gene). In isolating a cDNA highly homologous to a gene selected from OEX 1-242 from animals, it is particularly preferable to use tissues from ovarian endometrial cysts.

In place of hybridization, a gene amplification method, for example, the PCR method, can be utilized to isolate a DNA encoding a polypeptide having the biological activity of the OEX protein, using a primer synthesized based on the sequence information of the protein encoding DNA (OEX 1-242).

An OEX polypeptide used in the method of the present invention may have variations in amino acid sequence, molecular weight, isoelectric point, the presence or absence of sugar chains or form, depending on the cell or host used to produce it or the purification method utilized. Nevertheless, so long as it has a biological activity equivalent to that of the OEX protein, it may be used in the method of the present invention and such methods utilizing polypeptides with a biological activity equivalent to the OEX protein are within the scope of the present invention.

The OEX polypeptides used in the present invention can be prepared as recombinant proteins or natural proteins, by methods well known to those skilled in the art. A recombinant protein can be prepared by inserting a DNA, which encodes the OEX polypeptide, into an appropriate expression vector, introducing the vector into an appropriate host cell, obtaining the extract, and purifying the polypeptide. Alternatively, a natural protein can be isolated by methods known to a person skilled in the art, for example, by contacting the affinity column, in which antibodies binding to the OEX protein described below are bound, with the extract of tissues or cells expressing the OEX polypeptide. The antibodies can be polyclonal antibodies or monoclonal antibodies.

The OEX polypeptide to be contacted with a test compound can be, for example, a purified polypeptide, a soluble protein, a form bound to a carrier or a fusion protein fused with other polypeptides. Examples of supports that may be used for binding proteins include insoluble polysaccharides, such as agarose, cellulose and dextran; and synthetic resins, such as polyacrylamide, polystyrene and silicon; preferably commercial available beads and plates (multi-well plates, biosensor chip, etc.) prepared from the above materials may be used. When using beads, they may be filled into a column.

The binding of a protein to a support may be conducted according to routine methods, such as chemical bonding and physical adsorption. Alternatively, a protein may be bound to a support via antibodies that specifically recognizing the protein. Moreover, binding of a protein to a support can be also conducted by means of avidin and biotin binding.

As a method of screening for proteins, for example, that bind to the OEX polypeptide using any of the OEX polypeptides described above, many methods well known by a person skilled in the art can be used. Such a screening can be conducted by, for example, immunoprecipitation method, specifically, in the following manner.

In immunoprecipitation, an immune complex is formed by adding an antibody to cell lysate prepared using an appropriate detergent. The antibody used in the immunoprecipitation for the screening recognizes any of the proteins endcoded by OEX 1 -242. Alternatively, when an OEX protein fused with a recognition site (epitope) is used in the screening, antibodies against the epitope may be used for the immunoprecipitaion. The immune complex consists of the OEX protein, a polypeptide comprising the binding ability with the OEX protein, and an antibody.

An immune complex can be precipitated, for example by Protein A sepharose or Protein G sepharose when the antibody is a mouse IgG antibody. If the OEX polypeptide is prepared as a fusion protein with an epitope, such as GST, an immune complex can be formed in the same manner as in the use of the antibody against the OEX polypeptide, using a substance specifically binding to these epitopes, such as glutathione-Sepharose 4B.

Immunoprecipitation can be performed by following or according to, for example, the methods in the literature (Harlow and Lane (1988) Antibodies, 511-52, Cold Spring Harbor Laboratory publications, New York).

SDS-PAGE is commonly used for analysis of immunoprecipitated proteins and the bound protein can be analyzed by the molecular weight of the protein using gels with an appropriate concentration. Since the protein bound to the OEX polypeptide may be difficult to detect by a common staining method, such as Coomassie staining or silver staining, the detection sensitivity for the protein can be improved by culturing cells in culture medium containing radioactive isotope, ³⁵S-methionine or ³⁵S-cystein, labeling proteins in the cells, and detecting the proteins. The target protein can be purified directly from the SDS-polyacrylamide gel and its sequence can be determined, when the molecular weight of a protein has been revealed.

As a method for screening proteins binding to the OEX polypeptide using the polypeptide, for example, West-Western blotting analysis (Skolnik et al. (1991) Cell 65: 83-90) can be used. Specifically, a protein binding to the OEX polypeptide can be obtained by preparing a cDNA library from cells, tissues, organs or cultured cells expected to express a protein binding to the OEX polypeptide using a phage vector (e.g., ZAP), expressing the protein on LB-agarose, fixing the protein expressed on a filter, reacting the purified and labeled OEX polypeptide with the above filter, and detecting the plaques expressing proteins bound to the OEX polypeptide according to the label. The OEX polypeptide may be labeled by utilizing the binding between biotin and avidin, or by utilizing an antibody that specifically binds to the OEX polypeptide, or a peptide or polypeptide (for example, GST) that is fused to the OEX polypeptide. Methods using labeling substances such as radioisotope (³H, ¹⁴C, ³²P, ³³P, ³⁵S, ¹²¹I, ¹³¹I, etc.), enzymes (alkaline phosphatase, horseradish peroxidase, β-galactosidase, β-glucosidase, etc.), fluorescent substances (fluorescein isothiosyanete (FITC), rhodamine, etc.) and biotin/avidin, may be used for the labeling in the present method. When the OEX protein is labeled with radioisotope, the detection or measurement can be carried out by liquid scintillation. Alternatively, OEX proteins labeled with enzymes can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman (1992) Cell 68: 597-612”, “Fields and Sternglanz (1994) Trends Genet 10: 286-92”).

In the two-hybrid system, the OEX polypeptide is fused to the SRF-binding region or GAL4-binding region and expressed in yeast cells. A cDNA library is prepared from cells expected to express a protein binding to the OEX polypeptide, such that the library, when expressed, is fused to the VP16 or GAL4 transcriptional activation region. The cDNA library is then introduced into the above yeast cells and the cDNA derived from the library is isolated from the positive clones detected (when a protein binding to the OEX polypeptide is expressed in yeast cells, the binding of the two activates a reporter gene, making positive clones detectable). A protein encoded by the cDNA can be prepared by introducing the cDNA isolated above to E. coli and expressing the protein.

As a reporter gene, for example, Ade2 gene, lacZ gene, CAT gene, luciferase gene and such can be used besides HIS3 gene.

A compound binding to the OEX polypeptide can also be screened using affinity chromatography. For example, the OEX polypeptide may be immobilized on a carrier of an affinity column, and a test compound, containing a protein capable of binding to the OEX polypeptide, is applied to the column. A test compound herein may be, for example, cell extracts, cell lysates, etc. After loading the test compound, the column is washed, and compounds bound to the OEX polypeptide can be prepared.

When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon may be used as a mean for detecting or quantifying the bound compound in the present invention. When such a biosensor is used, the interaction between the OEX polypeptide and a test compound can be observed real-time as a surface plasmon resonance signal, using only a minute amount of polypeptide and without labeling (for example, BIAcore, Pharmacia). Therefore, it is possible to evaluate the binding between the OEX polypeptide and a test compound using a biosensor such as BIAcore.

The methods of screening for molecules that bind when the immobilized OEX polypeptide is exposed to synthetic chemical compounds, or natural substance banks, or a random phage peptide display library, or the methods of screening using high-throughput based on combinatorial chemistry techniques (Wrighton et al. (1996) Science 273: 458-64; Verdine (1996) Nature 384: 11-13; Hogan (1996) Nature 384: 17-9) to isolate not only proteins but chemical compounds that bind to the OEX protein (including agonist and antagonist) are well known to those skilled in the art.

Alternatively, when the biological activity of the OEX polypeptide is detected in the screening of the present invention, a compound isolated by this screening is a candidate for agonists or antagonists of the OEX polypeptide. The term “agonist” refers to molecules that activate the function of the OEX polypeptide by binding thereto. Likewise, the term “antagonist” refers to molecules that inhibit the function of the OEX polypeptide by binding thereto. Moreover, a compound isolated by this screening is a candidate for compounds which inhibit the in vivo interaction of the OEX polypeptide with molecules (including DNAs and proteins).

When the biological activity to be detected in the present method is cell proliferation, it can be detected, for example, by preparing cells which express the OEX polypeptide, culturing the cells in the presence of a test compound, and determining the speed of cell proliferation, measuring the cell cycle and such, as well as by measuring the colony forming activity.

A decrease in the binding activity or biological activity of one or more polypeptides encoded by OEX 1-97 and OEX 186-242 compared to a normal control level of the gene detected by the screening method indicates that the test compound is an inhibitor of the ovarian endometriosis-associated gene and is expected to reduce the symptom of endometriosis. Alternatively, an increase of the binding activity with or the biological activity of one or more polypeptides encoded by OEX 98-185 compared to a normal control level of the gene detected by the screening method indicates that the test compound is an enhancer of the ovarian endometriosis-associated gene and is expected to reduce the symptom of endometriosis. A compound isolated by the above screenings is a candidate for drugs which can be applied for the treatment or prevention of ovarian endometriosis. Moreover, compound in which a part of the structure of the compound that alters the activity of the OEX protein is converted by addition, deletion and/or replacement are also included in the compounds obtainable by the screening method of the present invention.

Any test compound, for example, cell extracts, cell culture supernatant, products of fermenting microorganism, extracts from marine organism, plant extracts, purified or crude proteins, peptides, non-peptide compounds, synthetic micromolecular compounds and natural compounds can be used in the screening methods of the present invention. The test compound of the present invention can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam (1997) Anticancer Drug Des. 12: 145). Examples of methods for the synthesis of molecular libraries can be found in the art (DeWitt et al. (1993) Proc. Natl. Acad. Sci. USA 90: 6909; Erb et al. (1994) Proc. Natl. Acad. Sci. USA 91: 11422; Zuckermann et al. (1994) J. Med. Chem. 37: 2678; Cho et al. (1993) Science 261: 1303; Carell et al. (1994) Angew. Chem. Int. Ed. Engl. 33: 2059; Carell et al. (1994) Angew. Chem. Int. Ed. Engl. 33: 2061; Gallop et al. (1994) J. Med. Chem. 37: 1233). Libraries of compounds may be presented in solution (see Houghten (1992) Bio/Techniques 13: 412) or on beads (Lam (1991) Nature 354: 82), chips (Fodor (1993) Nature 364: 555), bacteria (US Pat. No. 5,223,409), spores (US Pat. No. 5,571,698;5,403,484, and 5,223,409), plasmids (Cull et al. (1992) Proc. Natl. Acad. Sci. USA 89: 1865) or phage (Scott and Smith (1990) Science 249: 386; Delvin (1990) Science 249: 404; Cwirla et al. (1990) Proc. Natl. Acad. Sci. USA 87: 6378; Felici (1991) J. Mol. Biol. 222: 301; US Pat. Application 2002103360).

Selecting a Therapeutic Agent for Treating Ovarian Endometriosis Appropriate for a Particular Individual

Differences in the genetic makeup of individuals can result in differences in their relative abilities to metabolize various drugs. A compound that is metabolized in a subject to act as an anti-ovarian endometriosis agent can manifest itself by inducing a change in gene expression pattern in the subject's cells from that characteristic of an ovarian endometriosal state to a gene expression pattern characteristic of a non-ovarian endometriosal state. Accordingly, the differentially expressed OEX associated genes disclosed herein allow for selection of a putative therapeutic or prophylactic anti-ovarian endometriosis agent specifically adequate for a subject by testing candidate compounds in a test cell population from the selected subject.

To identify an anti-ovarian endometriosis agent, that is appropriate for a specific subject, a test cell or test cell population derived from the subject is exposed to a candidate therapeutic agent and the expression of one or more of OEX 1-242 genes is determined.

The test cell is or the test cell population contains an ovarian endometrial cell expressing an ovarian endometriosis associated gene. Preferably, the test cell is or the test cell population contains an epithelial cell. For example, a test cell or test cell population is incubated in the presence of a candidate agent and the pattern of gene expression in the test cell or cell population is measured and compared to one or more reference profiles (an ovarian endometriosis reference expression profile or a non-ovarian endometriosis reference expression profile).

A decrease in the expression of one or more of OEX 1-97 and 186-242 or an increase in the expression of one or more of OEX 98-185 in a test cell or test cell population relative to that in a reference cell population containing ovarian endometriosis is indicative that the agent is therapeutic.

The test agent can be any compound or composition. For example, the test agent is an immunomodulatory agent.

Kit

The present invention also provides a kit comprising any one or more of the materials (reagents and labware) used for performing the above-described method of diagnosing ovarian endometriosis. Preferably, the kit comprises an OEX-detection reagent, e.g., a nucleic acid that specifically binds to or identifies one or more of OEX nucleic acids. Such nucleic acid specifically binding to or identifying one or more of OEX nucleic acids are exemplified by oligonucleotide sequences that are complementary to a portion of an OEX nucleic acid. Alternatively, the kit comprises one or more antibodies which bind to polypeptides encoded by an OEX nucleic acid. The reagents are packaged together in the form of a kit. The reagents such as nucleic acids or antibodies (either bound to a solid matrix or packaged separately with reagents for binding them to the matrix), control reagents (positive and/or negative) and/or a means for detection of the nucleic acid or antibody are preferably packaged in separate containers.

For example, an OEX detection reagent is immobilized on a solid matrix such as a porous strip to form at least one OEX detection site. The measurement or detection region of the porous strip may include a plurality of detection sites, each detection site containing an OEX-detection reagent. A test strip may also contain sites for negative and/or positive controls. Alternatively, control sites are located on a separate strip from the test strip. Optionally, the different detection sites may contain different amounts of immobilized nucleic acids, i.e., a higher amount in the first detection site and lesser amounts in subsequent sites. Upon the addition of a test biological sample, the number of sites displaying a detectable signal provides a quantitative indication of the amount of OEX present in the sample. The detection sites may be configured in any suitably detectable shape and are typically in the shape of a bar or dot spanning the width of a teststrip.

Alternatively, the kit contains a nucleic acid substrate array comprising one or more ovarian endometriosis-associated genes. The nucleic acids on the array specifically identify one or more nucleic acid sequences represented by OEX 1-242. The expression level of 2, 3, 4, 5, 6, 7, 8, 9, 10, 15, 20, 25, 40 or 50 or more of the sequences represented by OEX 1-242 are identified by virtue if the level of binding to an array test strip or chip. The substrate array can be on a solid substrate, e.g., a “chip” as described in U.S. Pat. No.5,744,305.

The assay format of the kit is not restricted in any way, so long as an OEX nucleic acid or its gene product in a subject-derived biological sample can be detected with the use of the kit. Preferably, the kit is in the format of an immunoassay, encompassing any kind of immunoassay known in the art to detect a protein in a sample. For example, ELISA is a preferred method for performing the diagnosis of the present invention. A kit of the present invention for conducting ELISA preferably contains (1) a detectably labeled polyclonal antibody binding to an OEX polypeptide; and (2) a monoclonal antibody binding to the OEX polypeptide immobilized on a solid matrix. Alternatively, the kit may preferably contains (1) a detectably labeled polyclonal antibody binding to the OEX polypeptide; (2) a monoclonal antibody binding to the OEX polypeptide that can be immobilized on a solid matrix; and (3) a reagent for immobilizing the antibody of (2) on a matrix. The kit may further comprise a matrix for immobilizing the antibody of (2) with the reagent of (3).

A preservative such as Thimerosal may be added to the enzyme-labeled antibodies included in the kit. Further, stabilizers, such as glycerol, may be added. Labeled antibodies can be stored long term by subjecting to lyophilization and keeping them under cool and dark conditions. A preservative, such as sodium azide, may be added to the immobilized antibody and the antibody is preferably stored under a cool condition.

Furthermore, control samples (with a normal control level and/or ovarian endometriosis control level) may also be included in the kit of the present invention. The normal control sample can be obtained from a healthy subject and the endometriosis control sample also can be obtained from a subject which was diagnosed endometriosis. Alternatively, endometriosis control sample of the present invention can be prepared adding OEX protein to nomal control sample. Moreover, reagents (substrate, etc.) for detecting the labeled antibody are included in the kit. Preferably, instructions (written, tape, VCR, CD-ROM, etc.) for carrying out the assay may also be included in the kit.

Arrays and Pluralities

The invention also includes a nucleic acid substrate array comprising one or more ovarian endometriosis-associated genes. The nucleic acids on the array specifically corresponds to one or more nucleic acid sequences represented by OEX 1-242. The expression level of 2, 3, 4, 5, 6, 7, 8, 9, 10, 15, 20, 25, 40 or 50 or more of the OEX nucleic acids represented by OEX 1-242 are identified by detecting the binding of nucleotides to the array.

The invention also includes an isolated plurality of nucleic acids (i.e., a mixture if two or more OEX nucleic acids). The nucleic acids are in a liquid phase or a solid phase, e.g., immobilized on a solid support such as a nitrocellulose membrane. The plurality includes one or more of the nucleic acids represented by OEX 1-242. According to a further embodiment of the present invention, the plurality includes 2, 3, 4, 5, 6, 7, 8, 9, 10, 15, 20, 25, 40 or 50 or more of the nucleic acids represented by OEX 1-242.

Method of Treating or Preventing Ovarian Endometriosis

The invention provides a method for treating or preventing ovarian endometriosis in a subject. Therapeutic compounds are administered prophylactically or therapeutically to subject suffering from or at risk of (or susceptible to) developing endometriosis. Such subjects are identified using standard clinical methods or by detecting an aberrant expression level or activity of OEX 1-242. Prophylactic administration occurs prior to the manifestation of overt clinical symptoms of disease, such that a disease or disorder is prevented or, alternatively, delayed in its progression.

The therapeutic method includes increasing the expression or function, or both of one or more gene products of genes whose expression is decreased (“under-expressed genes”) in an ovarian endometrial cell relative to normal cells of the same tissue type from which the ovarian endometrial cells are derived. In these methods, the subject is treated with an effective amount of a compound, which increases the amount of one of more of the under-expressed genes (OEX 97-185) in the subject. Administration can be systemic or local. Therapeutic compounds include polypeptide products of the under-expressed gene, or a biologically active fragment thereof, a nucleic acid encoding an under-expressed gene downstream of expression control elements permitting expression of the gene in the ovarian endometrial cells, compounds that increase the expression level of such gene endogenously existing in the ovarian endometrial cells (i.e., compounds that up-regulate the expression of the under-expressed gene(s)). Administration of such therapeutic compounds counter the effects of aberrantly-under expressed gene(s) in the subject's ovarian cells and are expected to improve the clinical condition of the subject. Such compounds can be obtained by the screening method of the present invention described above.

The method also includes decreasing the expression or function, or both, of one or more gene products of genes whose expression is aberrantly increased (“over-expressed gene”) in ovarian endometrial cells. In these methods, the subject is treated with an effective amount of a compound, which decreases the amount of one of more of the over-expressed genes (OEX 1-96 and 186-242) in the subject. Administration can be systemic or local. Therapeutic compounds include compounds that decrease the expression level of such gene endogenously existing in the ovarian endometrial cells (i.e., compounds that down-regulate the expression of the over-expressed gene(s)). Administration of such therapeutic compounds counter the effects of aberrantly-over expressed gene(s) in the subjects ovarian cells and are expected to improve the clinical condition of the subject. Such compounds can be obtained by the screening method of the present invention described above.

The expression of over-expressed genes may be also inhibited in any of several ways known in the art including administering to the subject a nucleic acid that inhibits or antagonizes the expression of the over-expressed gene(s). Antisense oligonucleotides, siRNA or ribozymes which disrupts expression of the over-expressed gene(s) can be used for inhibiting the expression of over-expressed genes.

As noted above, antisense-oligonucleotides corresponding to any of the nucleotide sequence of OEX 1-97 or OEX 186-242 can be used to reduce the expression level of the OEX 1-97 or OEX 186-242. Antisense-oligonucleotides corresponding to OEX 1-97 or OEX 186-242 that are up-regulated in ovarian endometriosis are useful for the treatment or prevention of ovarian endometriosis. Specifically, the antisense-oligonucleotides of the present invention may act by binding to any of the polypeptides encoded by the OEX 1-97 or OEX 186-242, or mRNAs corresponding thereto, thereby inhibiting the transcription or translation of the genes, promoting the degradation of the mRNAs, and/or inhibiting the expression of proteins encoded by the OEX nucleotides, and finally inhibiting the function of the proteins. The term “antisense-oligonucleotides” as used herein encompasses both nucleotides that are entirely complementary to the target sequence and those having a mismatch of one or more nucleotides, so long as the antisense-oligonucleotides can specifically hybridize to the target sequence. For example, the antisense-oligonucleotides of the present invention include polynucleotides that have a homology of at least 70% or higher, preferably at 80% or higher, more preferably 90% or higher, even more preferably 95% or higher over a span of at least 15 continuous nucleotides to any of the nucleotide sequence of OEX 1-97 and OEX 186-242. Algorithms known in the art can be used to determine the homology. Furthermore, derivatives or modified products of the antisense-oligonucleotides can also be used as antisense-oligonucleotides in the present invention. Examples of such modified products include lower alkyl phosphonate modifications such as methyl-phosphonate-type or ethyl-phosphonate-type, phosphorothioate modifications and phosphoroamidate modifications.

The antisense-oligonucleotides and derivatives thereof act on cells producing the proteins encoded by marker genes (OEX 1-97, OEX 186-242) by binding to the DNAs or mRNAs encoding the proteins, inhibiting their transcription or translation, promoting the degradation of the mRNAs and inhibiting the expression of the proteins, thereby resulting in the inhibition of the protein function.

An antisense-oligonucleotides and derivatives thereof can be made into an external preparation, such as a liniment or a poultice, by mixing with a suitable base material which is inactive against the derivative.

The antisense-oligonucleotides of the invention inhibit the expression of at least one OEX protein encoded by any one of OEX 1-97 and OEX 186-242, and thus are useful for suppressing the biological activity of the protein.

The nucleic acids that inhibit one or more gene products of over-expressed genes also include small interfering RNAs (siRNA) comprising a combination of a sense strand nucleic acid and an antisense strand nucleic acid of the nucleotide sequence encoding an over-expressed OEX protein, such as OEX 1-97 and 186-242. The term “siRNA” refers to a double stranded RNA molecule which prevents translation of a target mRNA. Standard techniques of introducing siRNA into the cell can be used in the treatment or prevention of the present invention, including those in which DNA is a template from which RNA is transcribed. The siRNA is constructed such that a single transcript has both the sense and complementary antisense sequences from the target gene, e.g., a hairpin.

The method is used to suppress gene expression of a cell with up-regulated expression of an OEX gene. Binding of the siRNA to the OEX gene transcript in the target cell results in a reduction of OEX protein production by the cell. The length of the oligonucleotide is at least 10 nucleotides and may be as long as the naturally occurring transcript. Preferably, the oligonucleotide is 19-25 nucleotides in length. Most preferably, the oligonucleotide is less than 75, 50 or 25 nucleotides in length.

The nucleotide sequence of siRNAs may be designed using a siRNA design computer program available from the Ambion website (http://www.ambion.com/techlib/misc/siRNA_finder.html). Nucleotide sequences for the siRNA are selected by the computer program based on the following protocol:

Selection of siRNA Target Sites:

1. Beginning with the AUG start codon of transcript, scan downstream for AA dinucleotide sequences. Record the occurrence of each AA and the 3′ adjacent 19 nucleotides as potential siRNA target sites. Tuschl, et al. recommend not to design siRNA against the 5′ and 3′ untranslated regions (UTRs) and regions near the start codon (within 75 bases) as these may be richer in regulatory protein binding sites, and thus the complex of endonuclease and siRNAs that were designed against these regions may interfere with the binding of UTR-binding proteins and/or translation initiation complexes.

2. Compare the potential target sites to the human genome database and eliminate from consideration any target sequences with significant homology to other coding sequences. The homology search can be performed using BLAST, which can be found on the NCBI server at: www.ncbi.nlm.nih.gov/BLAST/

3. Select qualifying target sequences for synthesis. On the website of Ambion, several preferable target sequences can be selected along the length of the gene for evaluation.

The siRNAs inhibit the expression of over-expressed OEX protein and is thereby useful for suppressing the biological activity of the protein. Therefore, a composition comprising the siRNA is useful in treating or preventing ovarian endometriosis.

The nucleic acids that inhibit one or more gene products of over-expressed genes also include ribozymes against the over-expressed gene(s) (OEX 1-97 and OEX 186-242).

The ribozymes inhibit the expression of over-expressed OEX protein and is thereby useful for suppressing the biological activity of the protein. Therefore, a composition comprising the ribozyme is useful in treating or preventing ovarian endometriosis.

Generally, ribozymes are classified into large ribozymes and small ribozymes. A large ribozyme is known as an enzyme that cleaves the phosphate ester bond of nucleic acids. After the reaction with the large ribozyme, the reacted site consists of a 5′-phosphate and 3′-hydroxyl group. The large ribozyme is further classified into (1) group I intron RNA catalyzing transesterification at the 5′-splice site by guanosine; (2) group II intron RNA catalyzing self-splicing through a two step reaction via lariat structure; and (3) RNA component of the ribonuclease P that cleaves the tRNA precursor at the 5′ site through hydrolysis. On the other hand, small ribozymes have a smaller size (about 40 bp) compared to the large ribozymes and cleave RNAs to generate a 5′-hydroxyl group and a 2′-3′ cyclic phosphate. Hammerhead type ribozymes (Koizumi et al. (1988) FEBS Lett. 228: 225) and hairpin type ribozymes (Buzayan (1986) Nature 323: 349; Kikuchi and Sasaki (1992) Nucleic Acids Res. 19: 6751) are included in the small ribozymes. Methods for designing and constructing ribozymes are known in the art (see Koizumi et al. (1988) FEBS Lett. 228: 225; Koizumi et al. (1989) Nucleic Acids Res. 17: 7059; Kikuchi and Sasaki (1992) Nucleic Acids Res. 19: 6751) and ribozymes inhibiting the expression of an over-expressed OEX protein can be constructed based on the sequence information of the nucleotide sequence encoding the OEX protein according to conventional methods for producing ribozymes.

The ribozymes inhibit the expression of over-expressed OEX protein and is thereby useful for suppressing the biological activity of the protein. Therefore, a composition comprising the ribozyme is useful in treating or preventing ovarian endometriosis.

Alternatively, the function of one or more gene products of the over-expressed genes is inhibited by administering a compound that binds to or otherwise inhibits the function of the gene products. For example, the compound is an antibody which binds to the over-expressed gene product or gene products. The definition of the term “antibody” is as described above. A fragment of an antibody, as referred to hereinbefore, can also be used in the method of treating or preventing ovarian endometriosis of the present invention. An antibody may be modified by conjugation with a variety of molecules, such as polyethylene glycol (PEG). The modified antibody can be obtained by chemically modifying an antibody. These modification methods are conventional in the field.

Alternatively, an antibody may be obtained as a chimeric antibody, between a variable region derived from nonhuman antibody and the constant region derived from human antibody, or as a humanized antibody, comprising the complementarity determining region (CDR) derived from nonhuman antibody, the frame work region (FR) derived from human antibody, and the constant region. Such antibodies can be prepared using known technology.

The present invention provides a method for treating or preventing ovarian endometriosis, using an antibody against an over-expressed OEX polypeptide. According to the method, a pharmaceutically effective amount of an antibody against the OEX polypeptide is administered. An antibody against an over-expressed OEX polypeptide is administered at a dosage sufficient to reduce the activity of the OEX protein. Alternatively, an antibody binding to a cell surface marker specific for tumor cells can be used as a tool for drug delivery. Thus, for example, an antibody against an over-expressed OEX polypeptide conjugated with a cytotoxic agent may be administered at a dosage sufficient to injure tumor cells.

The present invention also relates to a method of treating or preventing ovarian endometriosis in a subject comprising administering to said subject a vaccine comprising a polypeptide encoded by a nucleic acid selected from the group consisting of OEX 1-97 and OEX 186-242 or an immunologically active fragment of said polypeptide, or a polynucleotide encoding the polypeptide or the fragment thereof. Administration of the polypeptide induces an anti-tumor immunity in a subject. The polypeptide or the immunologically active fragments thereof are useful as vaccines against ovarian endometriosis. An ovarian endometriosis which is benign tumor can be treated or prevented via inducing anti-tumor immunity in a subject. In some cases the proteins or fragments thereof may be administered in a form bound to the T cell receptor (TCR) or presented on an antigen presenting cell (APC), such as macrophage, dendritic cell (DC) or B-cells. Due to the strong antigen presenting ability of DC, the use of DC is most preferable among the APCs.

In the present invention, the phrase “vaccine against ovarian endometriosis” refers to a substance that has the function to induce anti-tumor immunity or immunity to suppress ovarian endometriosis upon inoculation into animals. In general, anti-tumor immunity includes immune responses such as follows:
induction of cytotoxic lymphocytes against tumors,
induction of antibodies that recognize tumors, and
induction of anti-tumor cytokine production.

Therefore, when a certain protein induces any one of these immune responses upon inoculation into an animal, the protein is decided to have anti-tumor immunity inducing effect. The induction of the anti-tumor immunity by a protein can be detected by observing in vivo or in vitro the response of the immune system in the host against the protein.

For example, a method for detecting the induction of cytotoxic T lymphocytes is well known. A foreign substance that enters the living body is presented to T cells and B cells by the action of antigen presenting cells (APCs). T cells that respond to the antigen presented by APC in antigen specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to T cell by APC, and detecting the induction of CTL. Furthermore, APC has the effect of activating CD4+ T cells, CD8+ T cells, macrophages, eosinophils and NK cells. Since CD4+ T cells and CD8+ T cells are also important in anti-tumor immunity, the anti-tumor immunity inducing action of the peptide can be evaluated using the activation effect of these cells as indicators.

A method for evaluating the inducing action of CTL using dendritic cells (DCs) as APC is well known in the art. DC is a representative APC having the strongest CTL inducing action among APCs. In this method, the test polypeptide is initially contacted with DC and then this DC is contacted with T cells. Detection of T cells having cytotoxic effects against the cells of interest after the contact with DC shows that the test polypeptide has an activity of inducing the cytotoxic T cells. Activity of CTL against tumors can be detected, for example, using the lysis of ⁵¹Cr-labeled tumor cells as the indicator. Alternatively, the method of evaluating the degree of tumor cell damage using ³H-thymidine uptake activity or LDH (lactose dehydrogenase)-release as the indicator is also well known.

Apart from DC, peripheral blood mononuclear cells (PBMCs) may also be used as the APC. The induction of CTL is reported to be enhanced by culturing PBMC in the presence of GM-CSF and IL-4. Similarly, CTL has been shown to be induced by culturing PBMC in the presence of keyhole limpet hemocyanin (KLH) and IL-7.

The test polypeptides confirmed to possess CTL inducing activity by these methods are polypeptides having DC activation effect and subsequent CTL inducing activity. Therefore, polypeptides that induce CTL against tumor cells are useful as vaccines against ovarian endometriosis. Furthermore, APC that acquired the ability to induce CTL against ovarian endometriosis by contacting with the polypeptides are useful as vaccines against ovarian endometriosis. Furthermore, CTL that acquired cytotoxicity due to presentation of the polypeptide antigens by APC can be also used as vaccines against ovarian endometriosis. Such therapeutic methods for ovarian endometriosis using anti-tumor immunity due to APC and CTL are referred to as cellular immunotherapy.

Generally, when using a polypeptide for cellular immunotherapy, efficiency of the CTL-induction is known to increase by combining a plurality of polypeptides having different structures and contacting them with DC. Therefore, when stimulating DC with protein fragments, it is advantageous to use a mixture of multiple types of fragments.

Alternatively, the induction of anti-tumor immunity by a polypeptide can be confirmed by observing the induction of antibody production against tumors. For example, when antibodies against a polypeptide are induced in a laboratory animal immunized with the polypeptide, and when growth, proliferation or metastasis of tumor cells is suppressed by those antibodies, the polypeptide can be determined to have an ability to induce anti-tumor immunity.

Anti-tumor immunity is induced by administering the vaccine of this invention, and the induction of anti-tumor immunity enables treatment and prevention of ovarian endometriosis. Therapy against or prevention of the onset of ovarian endometriosis includes any of the steps, such as inhibition of the growth of endometrial cyst cells, involution of endometrial cyst cells and suppression of occurrence of endometrial cyst cells. Decrease in mortality of individuals having ovarian endometriosis, decrease of endometriosis markers in the blood, alleviation of detectable symptoms accompanying ovarian endometriosis and such are also included in the therapy or prevention of ovarian endometriosis. Such therapeutic and preventive effects are preferably statistically significant. For example, in observation, at a significance level of 5% or less, wherein the therapeutic or preventive effect of a vaccine against ovarian endometriosis is compared to a control without vaccine administration. For example, Student's t-test, the Mann-Whitney U-test or ANOVA may be used for statistical analyses.

The above-mentioned protein having immunological activity, or a polynucleotide or vector encoding the protein may be combined with an adjuvant. An adjuvant refers to a compound that enhances the immune response against the protein when administered together (or successively) with the protein having immunological activity. Examples of adjuvants include cholera toxin, salmonella toxin, alum and such, but are not limited thereto. Furthermore, the vaccine of this invention may be combined appropriately with a pharmaceutically acceptable carrier. Examples of such carriers are sterilized water, physiological saline, phosphate buffer, culture fluid and such. Furthermore, the vaccine may contain as necessary, stabilizers, suspensions, preservatives, surfactants and such. The vaccine is administered systemically or locally. Vaccine administration may be performed by single administration or boosted by multiple administrations.

When using APC or CTL as the vaccine of this invention, ovarian endometriosis can be treated or prevented, for example, by the ex vivo method. More specifically, PBMCs of the subject receiving treatment or prevention are collected, the cells are contacted with the polypeptide ex vivo, and following the induction of APC or CTL, the cells may be administered to the subject. APC can be also induced by introducing a vector encoding the polypeptide into PBMCs ex vivo. APC or CTL induced in vitro can be cloned prior to administration. By cloning and growing cells having high activity of damaging target cells, cellular immunotherapy can be performed more effectively. Furthermore, APC and CTL isolated in this manner may be used for cellular immunotherapy not only against individuals from whom the cells are derived, but also against similar types of diseases in other individuals.

Pharmaceutical Compositions for Treating or Preventing Ovarian Endometriosis

The present invention provides compositions for treating or preventing ovarian endometriosis comprising a compound selected by the present method of screening for a compound that alters the expression or activity of an ovarian endometriosis-associated gene. Such therapeutic compositions are administered throughout the menstrual cycle or concordantly with a specific phase, e.g., proliferative or secretory phase of the cycle and may be administered to humans and other mammals, such as mice, rats, guinea-pig, rabbits, cats, dogs, sheep, pigs, cattle, monkeys, baboons or chimpanzees. Pharmaceutical formulations of the present compositions include those suitable for oral, rectal, nasal, topical (including buccal and sub-lingual), vaginal or parenteral (including intramuscular, sub-cutaneous and intravenous) administration, or for administration by inhalation or insufflation. The formulations are optionally packaged in discrete dosage units.

Pharmaceutical formulations suitable for oral administration include capsules, cachets or tablets, each containing a predetermined amount of the active ingredient. Formulations also include powders, granules, solutions, suspensions or emulsions. The active ingredient is optionally administered as a bolus electuary or paste. Tablets and capsules for oral administration may contain conventional excipients such as binding agents, fillers, lubricants, disintegrant or wetting agents. A tablet may be made by compression or molding, optionally with one or more formulational ingredients. Compressed tablets may be prepared by compressing in a suitable machine the active ingredients in a free-flowing form such as a powder or granules, optionally mixed with a binder, lubricant, inert diluent, lubricating, surface active or dispersing agent. Molded tablets may be made via molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for reconstitution with water or other suitable vehicle prior to use. Such liquid preparations may contain conventional additives such as suspending agents, emulsifying agents, non-aqueous vehicles (which may include edible oils) or preservatives. The tablets may optionally be formulated so as to provide slow or controlled release of the active ingredient in vivo. A package of tablets may contain one tablet to be taken on each of the month. The formulation or does of medicament varies with respect to the phase (proliferative or secretory) of the menstrual cycle.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration include suppositories with standard carriers such as cocoa butter or polyethylene glycol. Formulations for topical administration in the mouth, for example, buccally or sublingually, include lozenges, which contain the active ingredient in a flavored base such as sucrose and acacia or tragacanth, and pastilles comprising the active ingredient in a base such as gelatin, glycerin, sucrose or acacia. For intra-nasal administration of an active ingredient, a liquid spray or dispersible powder or in the form of drops may be used. Drops may be formulated with an aqueous or non-aqueous base also comprising one or more dispersing agents, solubilizing agents or suspending agents.

For administration by inhalation the compositions are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichiorotetrafluoroethane, carbon dioxide or other suitable gas. In the case of a pressurized aerosol, the dosage unit may be determined by providing a valve to deliver a metered amount.

Alternatively, for administration by inhalation or insufflation, the compositions may take the form of a dry powder composition, for example, a powder mix of an active ingredient and a suitable powder base such as lactose or starch. The powder composition may be presented in unit dosage form in, for example, capsules, cartridges, gelatin or blister packs from which the powder may be administered with the aid of an inhalator or insufflators.

Other formulations include implantable devices and adhesive patches; which release a therapeutic agent.

When desired, the above described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents.

Preferred unit dosage formulations are those containing an effective dose, as recited below, of the active ingredient or an appropriate fraction thereof.

For each of the aforementioned conditions, the compositions, e.g., polypeptides and organic compounds are administered orally or via injection at a dose of from about 0.1 to about 250 mg/kg per day. The dose range for adult humans is generally from about 5 mg to about 17.5 g/day, preferably about 5 mg to about 10 g/day, and most preferably about 100 mg to about 3 g/day. Tablets or other unit dosage forms of presentation provided in discrete units may conveniently contain an amount which is effective at such dosage or as a multiple of the same, for instance, units containing about 5 mg to about 500 mg, usually from about 100 mg to about 500 mg.

The dose employed will depend upon a number of factors, including the age and sex of the subject, the precise disorder being treated, and its severity. Also the route of administration may vary depending upon the condition and its severity.

The present invention further provides a composition for treating or preventing ovarian endometriosis comprising active ingredient that inhibits the expression of any one of the gene selected from the group of OEX 1-97 or OEX 186-242. Such active ingredient can be an antisense-oligonucleotide, siRNA or ribozyme against the gene, or derivatives, such as expression vector, of the antisense-oligonucleotide, siRNA or ribozyme. The active ingredient may be made into an external preparation, such as liniment or a poultice, by mixing with a suitable base material which is inactive against the derivatives.

Also, as needed, the active ingredient can be formulated into tablets, powders, granules, capsules, liposome capsules, injections, solutions, nose-drops and freeze-drying agents by adding excipients, isotonic agents, solubilizers, preservatives, pain-killers and such. These can be prepared according to conventional methods for preparing nucleic acid containing pharmaceuticals.

Preferably, the antisense-oligonucleotide derivative, siRNA derivative or ribozyme derivative is given to the patient by direct application to the ailing site or by injection into a blood vessel so that it will reach the site of ailment. A mounting medium can also be used in the composition to increase durability and membrane-permiability. Examples of mounting mediums include liposome, poly-L-lysine, lipid, cholesterol, lipofectin and derivatives thereof.

The dosage of such compositions can be adjusted suitably according to the patient's condition and used in desired amounts. For example, a dose range of 0.1 to 100 mg/kg, preferably 0.1 to 50 mg/kg can be administered.

Another embodiment of the present invention is a composition for treating or preventing ovarian endometriosis comprising an antibody against a polypeptide encoded by any one of the genes selected from the group of OEX 1-97 and OEX 186-242 or fragments of the antibody that bind to the polypeptide.

Although there are some differences according to the symptoms, the dose of an antibody or fragments thereof for treating or preventing ovarian endometriosis is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the condition of the patient, symptoms of the disease and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kg of body-weight.

### INDUSTRIAL APPLICABILITY

The gene-expression analysis of ovarian endometriosis described herein, obtained through a combination of laser-capture dissection and genome-wide cDNA microarray, has identified specific genes as targets for prevention and therapy of ovarian endometriosis. Based on the expression of a subset of these differentially expressed genes, the present invention provides molecular diagnostic markers for identifying or detecting ovarian endometriosis.

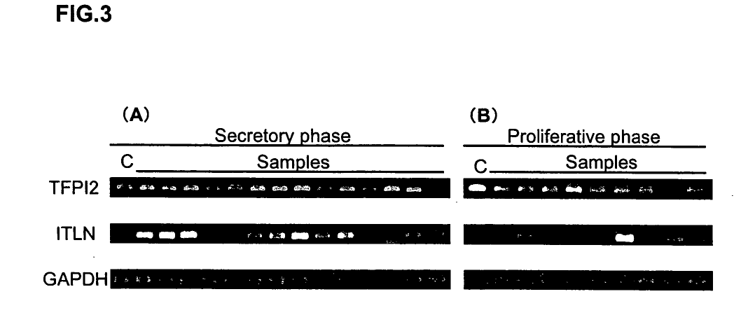

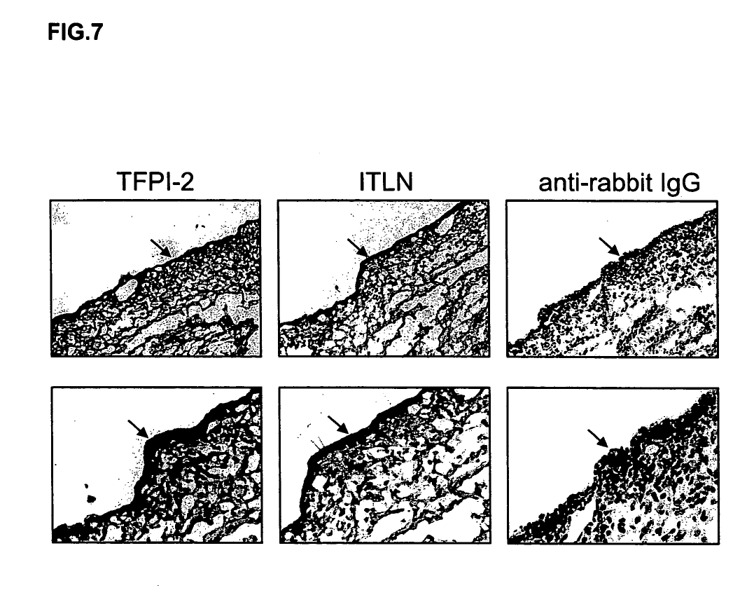

Various gynecological studies about TFPI-2 have been performed in terms of its association with the progression of pregnancy. However, to date, there has been no report that this protein relates to the development of endometriosis. Further, little is known whether ITLN participates in the progression or maintenance of various gynecologic disorders or tumor disease. Accordingly, the present inventors examined whether TFPI-2 and ITLN are secreted from cells through the establishment of stable transformants of each gene, and demonstrated that these genes were overexpressed in endometrial cysts by semi-quantitative RT-PCR and immunohistochemistry (FIG. 3 and 7).

As discussed in detail below in the Examples section, the present invention demonstrates the possible involvement of the TFPI-2 and ITLN proteins in human ovarian endometriosis and that TFPI-2 level was elevated in the sera of endometriosis patients as compared to normal controls. Since the expression of these transcripts is relatively low in normal human adult tissues, these genes themselves can serve as novel targets for therapy.

The methods described herein are also useful in the identification of additional molecular targets for prevention, diagnosis and treatment of ovarian endometriosis. The data reported herein add to a comprehensive understanding of ovarian endometriosis, facilitate development of novel diagnostic strategies and provide clues for identification of molecular targets for therapeutic drugs and preventative agents. Such information contributes to a more profound understanding of ovarian endometriosis, and provides indicators for developing novel strategies for diagnosis, treatment and ultimately prevention of ovarian endometriosis.

### EXAMPLES

The following examples are presented to illustrate the present invention and to assist one of ordinary skill in making and using the same. The examples are not intended in any way to otherwise limit the scope of the invention.

### Example 1

### Materials and General Methods

1. Tissue Preparation

Endometrial cysts were obtained after informed pre-operative consent from 6 patients who underwent cystectomy. For immunohistochemistry, tissue sections were mounted in O.C.T compound (Sakura Finetechnical) immediately after resection.

2. Sera

Sera were obtained from 36 patients one day before laparoscopic operation. Two of the patients were classified as in the rASRM stage I, 13 in stage III and 21 in stage IV. As controls, serum samples were collected from 12 apparently healthy women and eight men between the ages of 24 to 40.

3. Semi-Quantitative RT-PCR

Semi-quantitative RT-PCR experiments were conducted as described previously (Ono et al. (2000) Cancer Res. 60: 5007-11). Endometrial cysts were obtained to extract total RNA, and T7-based RNA amplification with the total RNA was performed according to a previous study (Arimoto et al. (2003) Int. J. Oncol. 22: 551-60). A 3-μg aliquot of amplified-RNA from each sample was reverse-transcribed for single-stranded cDNAs using random primer (Roche) and Superscript II (Life Technologies, Inc.). Each cDNA mixture was diluted for subsequent PCR amplification with the following primer sets

| | |
|---|---|
| TFPI-2 forward primer: | |
| 5′-TGACAGCATGAGGAAACAAATC-3′ | (SEQ ID NO: 23) |
| and | |
| reverse primer: | |
| 5′-ACGACCCCAAGA AATGAGTG-3′; | (SEQ ID NO: 24) |
| ITLN forward primer: | |
| 5′-GCATTGGTGGAGGAGGATAC-3′ | (SEQ.ID.NO.25) |
| and | |
| reverse primer: | |
| 5′-TGCCATTAACATTCTAGCTACTGG-3′; | (SEQ.ID.NO.26) |
| G3PDH forward primer: | |
| 5′-CGACCACTTTGTCAAGCTCA-3′ | (SEQ ID NO: 21) |
| and | |
| reverse primer: | |
| 5′-GGTTGAGC ACAGGGTACTTTATT-3′. | (SEQ ID NO: 22)) |

The expression of G3PDH served as an internal control. PCR reactions were optimized for the number of cycles to ensure product intensity within the linear phase of amplification.

4. Northern Blotting

Multiple-tissue Northern blot membranes containing 2-μg of poly(A)⁺ RNA from various human tissues (Clontech) were hybridized with ³²P-labeled partial cDNA fragments of TFPI-2 or ITLN (TFPI-2 forward primer: 5′-GGAAAATTCGGAAGAAGCAA-3′ (SEQ ID NO: 27) and reverse primer: 5′-ACGACCCCAA GAAATGAGTG-3′ (SEQ ID NO: 24); ITLN forward primer: 5′-CGGGATFTTGTTCAGTTCAGG-3′ (SEQ ID NO: 28) and reverse primer: 5′-TGCCATTAACATTCTAGCTACTGG-3′ (SEQ ID NO: 26)). Conditions of hybridization and washing are described elsewhere (Nakagawa et al. (2000) Oncogene 19: 210-6).

5. Recombinant Protein Production in Escherichia Coli and Polyclonal Antibody Generation

Recombinant proteins were prepared from human cDNA clones encoding TFPI-2 (GenBank Accession No. D29992) or ITLN (GenBank Accession No. BC020664) without their signal peptides (TFPI-2: N-terminal 22 residues, ITLN: N-terminal 18 residues) by inserting into the E. coli expression vector pET28a (Novagen) and transforming into BL21 -CodonPlus® (DE3)-RIL competent cells (Stratagene). Protein expression was induced by 0.5 mM isopropyl β-D-thiogalactoside (IPTG) with incubation at 37° C. for 3 h, and cells were harvested by centrifugation. E. coli cell pellets that expressed recombinant TFPI-2 or ITLN was dissolved in 100 mM sodium phosphate dehydrate (pH 8.0) containing 6M guanidine hydrochloride, 10 mM Tris and 10 mM imidazole. Histidine-tagged TFPI-2 or ITLN protein was purified by BD TALON™ Metal Affinity Resins (BD Biosciences). Then, 6 M guanidine hydrochloride was replaced with 8M urea. These purified recombinant proteins were refolded by dilution and reducing the concentration of urea to 4M (TFPI-2) or 2.5M (ITLN). In addition, ITLN protein was further purified by anion-exchanged high performance liquid chromatography using a Mono-Q HR5/5 column (Amersham Biosciences) with ÄKTAexplorer 10S (Amersham Biosciences). These protein solutions were injected into rabbits every week, and after 10 times of immunization, antiserum was collected (MBL). The specific antibodies were isolated by affinity chromatography using Affi-Gel 10 (for TFPI-2, Bio-Rad) or Affi-Gel 15 (for ITLN, Bio-Rad) to which purified recombinant protein is bound covalently.

6. Cell Lines

Human endometrial adenocarcinoma cell line, HEC-151, was provided by Kitasato University (Sagamihara, Japan) and was maintained in Eagle's minimal essential medium with 10% fetal bovine serum (FBS). Human glioma cell line, Hs.683, was purchased from the American Type Culture Collection (Manassas, Va., USA). Hs.683 and COS-7 cells were maintained in Dulbecco's modified Eagle's medium with 10% FBS. Cells were maintained at 37° C. in an atmosphere of humidified air with 5% CO₂.

7. Western Blotting

Samples were resolved by SDS-PAGE under reducing conditions and transferred onto Hybond™ ECL™ Nitrocellulose membranes (Amersham Pharmacia Biotech). The blotted membranes were blocked with Block Ace™ powder (Dainippon Seiyaku) and treated with rabbit anti-TFPI-2 (0.34 μg/ml) or anti-ITLN (0.16 μg/ml) specific polyclonal antibodies. After washing, the blots were treated with horseradish peroxidase-conjugated donkey anti-rabbit IgG (Amersham Biosciences) and developed with enhanced chemilumine scence (ECL; Amersham Biosciences).

8. Immunofluorescent Staining

Cells were replated on Lab-Tek® II Chamber Slide System (Nalge Nunc International) followed by fixation with 4% paraformaldehyde in PBS and permeabilization with 0.1% Triton X-100 in PBS for 3 min at 4° C. After blocking with 3% BSA in PBS for 1 h at room temperature, the cells were incubated with rabbit anti-TFPI-2 (0.34 μg/ml) or anti-ITLN (0.16 μg/ml) antibodies for 1 h at room temperature. These antibodies were stained with goat anti-rabbit secondary antibody conjugated to rhodamine, respectively, and viewed with a BX51 microscope (Olympus). As described in the following stable transformants section, stable transformants were also incubated with mouse anti-myc 9E10 monoclonal antibody (Santa Cruz Biotechnology, 0.2 μg/ml) and stained with rabbit anti-mouse secondary antibody conjugated to FITC.

9. Immunohistochemical Staining and Cross-Inhibition Assay

Archieved, paraformaldehyde-fixed, paraffin-embedded tissue sections were purchased from Biochain Institute, Inc. Sections were stained with DAKO EnVision™+System, HRP (DAB) (Dako) according to manufacturer's protocol. The polyclonal antibodies were used at 2.5 μg/ml (anti-TFPI-2 antibody) or at 2 μg/ml (anti-ITLN antibody). For immunostaining inhibition, antibodies were pre-incubated overnight at 4° C. with the corresponding recombinant proteins that were used as the antigen (1.5 μg).

10. Construction of Stable Transformants

COS-7 cells were seeded and transfected with pcDNA3.1/myc-His© (−)-TFPI-2 (Invitrogen) or pcDNA3.1/myc-His© (−)-ITLN (Invitrogen), or with an empty vector as a control, using FuGENE6 Transfection Reagent (Roche) according to the manufacturer's protocol. Cells were cultured for up to 3 weeks in the medium containing 0.4mg/ml of G418. Individual clones were isolated with cloning cylinders. The cell clones that expressed TFPI-2 or ITLN (as confirmed by RT-PCR, Western blotting, and immunofluorescent staining) were maintained in the medium containing 0.4 mg/ml of G418 and used for further investigations.

11. Measurement of TFPI-2 Concentration

The concentration of TFPI-2 was measured by ELISA using biotinylated anti-TFPI-2 polyclonal antibody. First, 100 μl rabbit anti-TFPI-2 antibody (10 μg/ml in 50 mmol/l sodium carbonate, pH 9.5) was added to each well of 96-well microtitration plates (Maxisorp Immunoplate, Nunc) and incubated overnight at 4° C. After washing the plates three times with PBS-0.1% Tween 20, each of the wells was blocked with 200 μl PBS-2% BSA at 37° C. for 2 h. Then, the plates were washed again three times with PBS-Tween 20. Subsequently, a 100 μl serum sample was added to each of the wells and the plates were incubated at 37° C. for 2 h. The plates were then washed three times with PBS-Tween 20 and 100 μl biotin-labeled anti-TFPI-2 antibody (1 μg/ml in PBS-1% BSA) was added to each of the wells. Biotinylation of the antibody was performed using ECL Protein Biotinylation Module (Amersham Biosciences) according to the manufacturer's protocol. After incubation for 2 h at 37° C., the plates were washed three times with PBS-Tween 20 and then treated with 100 μl peroxidase-conjugated avidin (DAKO, diluted 4000-fold with PBS-1% BSA) for 2 h at room temperature. Then, the plates were washed six times with PBS-Tween 20 and 100 μl o-phenylenediamine (DAKO, 4 tablets in 12 ml distilled water and 5 μl of 30% hydrogen peroxide) was added to each of the wells. After incubating the plates for an adequate time (3 to 5 min) at room temperature, 100 μl of 0.5 mol/l surfuric acid was added to each of the wells and the absorbance (A₄₉₀) was measured. The concentration of TFPI-2 in samples was interpolated from standard curves of A₄₉₀ versus recombinant TFPI-2 concentration.

### Example 2

### Correlation of Clinicopathological Features with Differential Gene Expression in Endometrial Tissue of the Patient

Endometrial cysts were obtained after informed pre-operative consent from 23 patients (women) who underwent cystectomy. Relevant clinical features of these patients are summarized in Table 10 below.

| | | | Days after | |
|---|---|---|---|---|
| | | | last menstrual | Cyst |
| Case No. | Age | Phase | period | size (cm) |
| 6 | 35 | proliferative | 9 | 2, 9 |
| 15 | 38 | proliferative | 10 | 4 |
| 19 | 44 | proliferative | 10 | 7 |
| 21 | 25 | proliferative | 1 | 5 |
| 22 | 37 | proliferative | 7 | 5, 7 |
| 302 | 35 | proliferative | 13 | 8 |
| 303 | 35 | proliferative | 15 | 6, 4 |
| 304 | 25 | proliferative | 9 | 6, 4 |
| 305 | 31 | proliferative | 4 | 7 |
| 3 | 37 | secretory | 21 | 5 |
| 7 | 32 | secretory | 23 | 8 |
| 11 | 23 | secretory | 19 | 6 |
| 12 | 29 | secretory | 31 | 7 |
| 14 | 41 | secretory | 12 | 8, 2 |
| 16 | 44 | secretory | 13 | 6 |
| 18 | 39 | secretory | 22 | 4 |
| 101 | 41 | secretory | 15 | 5 |
| 102 | 37 | secretory | 24 | 6 |
| 104 | 35 | secretory | 15 | 6 |
| 105 | 31 | secretory | 37 | 5, 6 |
| 106 | 31 | secretory | 35 | 6 |
| 107 | 30 | secretory | 30 | 3 |
| 151 | 38 | secretory | 25 | 9 |

Two cysts for the samples in case No. 6, 22, 303, 304, 14 and 105 were used.

The ages of the patients ranged from 23 to 44, and none had been on hormonal therapy within two years prior to the surgery. Nine patients were in the proliferative phase of the menstrual cycle at the time of surgery and the other 14 were in the secretory phase. Cysts were histopathologically diagnosed according to standard methods. As control samples, eutopic endometrial tissues were obtained by dilatation and curettage from the uteri of 14 of the patients, seven in the proliferative phase and the other seven in the secretory phase. Epithelial cells were scraped immediately after resection and suspended in ice-cold Dulbecco's modified Eagle's medium (DMEM: Sigma) supplemented with 10% fetal calf serum (FCS), then separated from interstitial cells under a stereomicroscope, and their purity was confirmed under a phase-contrast microscope as described previously (Jimbo et al. (1997) Am. J. Pathol. 150: 1173-8). Isolation of epithelial cells was as described elsewhere (Homung et al. (1998) Fertil. Steril. 69: 909-15; Sugawara et al. (1997) Biol. Reprod. 57: 936-42; Zhang et al. (1995) J. Cell Sci. 108: 323-32) with some modifications. Endometrial tissue was minced into small pieces and digested with 0.25% collagenase (Sigma) for 1 hour at 37° C. Using serial filtration, tissue debris was separated with 100-mm nylon sieves (Falcon) to remove mucus and undigested tissue. Then each filtrate was passed through a 40-mm nylon sieve that allowed stromal cells to go through. Epithelial glands were backwashed onto tissue culture dishes with DMEM/10%FCS and incubated at 37° C. for 30 minutes in 5% CO₂ to attach fibroblasts on the dishes. Epithelial cells were recovered in the supernatant and used for extraction of total RNA.

### Example 3

### Identification of Endometriosis-Associated Genes

Tissue obtained from diseased tissue (epithelial cells from endometriosis cysts) and normal tissues was evaluated to identify genes which are differently expressed in a disease state (endometriosis). The assays were carried out as follows.

1. RNA Preparation and T7-Based RNA Amplification

Total RNA was extracted by suspending the epithelial cells in RNA lysis buffer (RLT buffer, QIAGEN Inc.) and purifying according to the manufacturer's instructions. After treatment with DNase I (Nippon Gene, Tokyo Japan), T7-based amplification was carried out according to known methods. Three rounds of amplification were performed to obtain sufficient amounts of amplified RNA (aRNA). Control samples were amplified in two rounds. Two kinds of universal control were prepared, one the eutopic aRNA mixture derived from seven patients in the proliferative phase and the other from seven patients in the secretory phase.

RNA amplified by this method accurately reflected the proportions in the original RNA source. This correspondence had been confirmed earlier by semi-quantitative reverse transcription-polymerase chain reaction (RT-PCR) experiments. As a result, data from microarrays proved to be consistent with results obtained by RT-PCR regardless whether the total RNA or aRNA was used as the template (Ono et al. (2000) Cancer Res. 60: 5007-11).

2. Preparation of the Microarray

To obtain cDNAs for spotting on the glass slides, RT-PCR was performed for each gene as described previously (Okabe et al. (2001) Cancer Res. 61: 2129-2137). The PCR products were spotted on type 7 glass slides (Amersham Biosciences) with a Microarray Spotter Generation III (Amersham Biosciences). 4,608 genes were spotted in duplicate on each slide. Five different sets of slides were prepared (in total 23,040 genes), on each of which the same 52 housekeeping genes and two negative-control genes were spotted as well. Then 2.5-μg aliquots of aRNA from eutopic endometrial tissues and the corresponding ovarian endometrial cysts were labeled respectively with Cy3-dCTP and Cy5-dCTP (Amersham Biosciences). Hybridization, scanning and quantification of signals were performed as described previously (Ono et al. (2000) Cancer Res. 60: 5007-11) except that all processes were carried out with an Automated Slide Processor (Okabe et al. (2001) Cancer Res. 61: 2129-2137). The fluorescence intensities of Cy5 and Cy3 for each target spot were adjusted so that the mean Cy5/Cy3 ratios of 52 housekeeping genes were equal to one. Because data derived from low signal intensities are not very reliable, cut-off values for signal intensities on each slide were first determined to exclude genes for further analysis when both Cy3 and Cy5 dyes gave signal intensities lower than the cut-off. The relative expression of each gene (Cy5/Cy3 intensity ratio) was defined into one of four categories: up-regulated (ratio≧2.0), down-regulated (ratio≦0.5), unchanged (0.5<ratio<2.0) and not expressed (under the cutoff level of detection). Furthermore, the relative expression of each gene (Cy5/Cy3 intensity ratio) was defined into one of three additional categories: five fold up-regulated (ratio≧5.0). 0.2 fold down-regulated (ratio≦0.2) and 0.2-5 unchanged (0.2<ratio, <5.0).

3. Semi-Quantitative RT-PCR

The representative 10 commonly up-regulated genes throughout the menstrual cycle were selected and examined for their expression levels by the semi-quantitative RT-PCR experiments. A 3-μg aliquot of aRNA from each sample was reverse-transcribed for single-stranded cDNAs using random primer (Roche) and Superscript II (Life Technologies, Inc.). Each cDNA mixture was diluted for subsequent PCR amplification with the same primer sets that were prepared for the target DNA- or G3PDH-specific reactions. The primer sequences are listed in Table 11.

| | | |
|---|---|---|
| Genbank | | |
| Accession | | SEQ ID |
| No. | Abbreviation | NO: |

| | | | |
|---|---|---|---|
| | | Forward primer | |
| M63262 | ALOX5AP | 5′-TGGGGTTGGTGTTCTCAT | 1 |
| | | CT-3′ | |
| AA583491 | HCA112 | 5′-TGGAATCTAGCCATGCCT | 3 |
| | | CT-3′ | |
| X00457 | HLA-DPA1 | 5′-CTGAACTCCAGCTGCCCT | 5 |
| | | AC-3′ | |
| K01505 | HLA-DQA1 | 5′-ATCGCCATCTACAGGAGC | 7 |
| | | AG-3′ | |
| X63629 | CDH3 | 5′-ACCTTCTTAGGCCTCCTG | 9 |
| | | GT-3′ | |
| X00637 | HP | 5′-CTGGTATGCGACTGGGAT | 11 |
| | | CT-3′ | |
| M81141 | HLA-DQB1 | 5′-TCCTGCACTGACTCCTGA | 13 |
| | | GA-3′ | |
| M86511 | CD14 | 5′-CCGAGGTGGATAACCTGA | 15 |
| | | CA-3′ | |
| M32093 | | 5′-CTCACACATTGCGAACAA | 17 |
| | | CA-3′ | |
| AI310156 | | 5′-GCCTCACAAAAGAGCCAG | 19 |
| | | AG-3′ | |
| M33197 | G3PDH | 5′-CGACCACTTTGTCAAGCT | 21 |
| | | CA-3′ | |
| | | Reverse Primer | |
| M63262 | ALOX5AP | 5′-ACCTGGTCACAAAACATC | 2 |
| | | TTCAG-3′ | |
| AA583491 | HCA112 | 5′-ATCACATGACTACTCAGG | 4 |
| | | AGGGG-3′ | |
| X00457 | HLA-DPA1 | 5′-GGAGAACAGAGGATAAAA | 6 |
| | | GGCTC-3′ | |
| K01505 | HLA-DQA1 | 5′-CCAGGCATGTCTTTGTAG | 8 |
| | | GTAAC-3′ | |
| X63629 | CDH3 | 5′-TACACGATTGTCCTCACC | 10 |
| | | CTTC-3′ | |
| X00637 | HP | 5′-TGATTGACTCAGCAATGC | 12 |
| | | AGG-3′ | |
| M81141 | HLA-DQB | 5′-GAATAGAAACAGAAACCC | 14 |
| | | CTTGG-3′ | |
| M86511 | CD14 | 5′-GAATTGGTCGAAAAGTCC | 16 |
| | | TCAAC-3′ | |
| M32093 | | 5′-ATGGTGCTTTTAAGAAGA | 18 |
| | | GAGCC-3′ | |
| AI310156 | | 5′-GATCCACATTGGTGTTAC | 20 |
| | | CAGTT-3′ | |
| M33197 | G3PDH | 5′-GGTTGAGCACAGGGTACT | 22 |
| | | TTATT-3′ | |

Accession numbers and gene symbols were retrieved from the Unigene Databases (build#131).

The expression of G3PDH served as an internal control. PCR reactions were optimized for the number of cycles to ensure product intensity within the linear phase of amplification.

### Example 4

### Identification of Genes with Clinically Relevant Expression Patterns in Ovarian Endometrial Cells

Gene-expression profiles of epithelial cells from ovarian endometrial cysts were analyzed using a comprehensive cDNA microarray system containing 23,040 genes. Individual data were excluded when Cy5 and Cy3 signals were under cut-off values (see Example 1). Then the expression levels in cysts from nine patients in the proliferative phase of the menstrual cycle were compared with the expression level in a universal control consisting of a mixture of eutopic endometrial cells from seven women in the same phase. For examining gene expression during the secretory phase, 14 relevant cysts were compared with a mixture of eutopic endometrial cells from seven women in that phase. When cut-off value of 2.0 was applied for the signal-intensity ratios of Cy5/Cy3, 15 genes including two expressed-tag sequences (ESTs) were up-regulated at least 70% of the 23 cases (Table 1). The protocol selected 42 genes including 15 ESTs as up-regulated only in the proliferative phase (Table 2), and 40 genes including 10 ESTs only in the secretory phase (Table 3). Most of the genes listed in Table 2 were also over-expressed in the secretory phase and most of the genes listed in Table 3 were also over-expressed in the proliferative phase, but both in fewer than 70% of the cases. However, some genes were up-regulated in exclusively in one phase or the other: S100 calcium-binding protein A13 (S100A13), myosin regulatory light chain 2 and smooth muscle isoform (MYRL2) were up-regulated specifically in the proliferative phase, while genes encoding four and a half LIM domains 2 (FHL2) and TSPY-like (TSPYL) were up-regulated in only the secretory phase. Many ectopic endometria histopathologically show proliferative features when their corresponding eutopic endometrial tissues are in the secretory phase (Mathur et al. (1990) Fertil. Steril. 54: 56-63; Molitor (1971) Am. J. Obstet. Gynecol. 110: 275-84). Endometrial cells might continue to proliferate throughout the menstrual cycle when the expression of some genes is altered.

Experiments also revealed down-regulation of numerous genes in the cysts. Three hundred thirty-seven genes, including one hundred sixty-four ESTs, were commonly under-expressed in more than 70% of the patients during either proliferative or secretory phases when the cut-off signal intensity ratio of Cy5/Cy3 was set to 0.5. One hundred forty-four other genes were selected, including forty-one ESTs, as being under-expressed only in the proliferative phase, and eight hundred thirty-five genes including four hundred twenty-eight ESTs only in the secretory phase. Among them, only the genes listed in Tables 4, 5 and 6 are those whose Cy5/Cy3 signal intensity ratios were less than 0.3 in more than 70% of the cases examined.

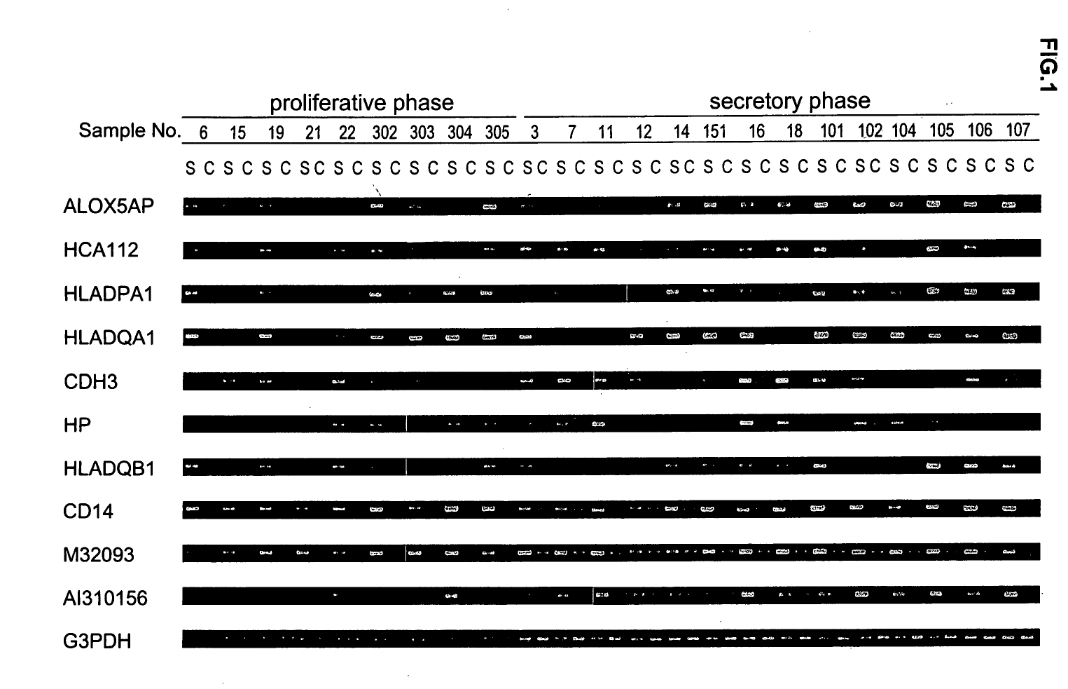

Semi-quantitative RT-PCR experiments were performed to confirm the differences in expression indicated by microarray analysis. By comparing the results with ratios of expression levels of the representative ten genes that were up-regulated throughout the menstrual cycle, good correspondence was verified with the microarray analysis in the great majority of cases tested (FIG. 1).

Many of the up-regulated genes encode elements of the immune system. For example, genes encoding histocompatibility proteins HLA-DPA1, HLA-DQA1, HLA-DQB1, HLA-DRA, HLA-DRB1 and CD14 were all over-expressed throughout the menstrual cycle. In addition, genes encoding complement factors C3, BF, C1S, C1R and C2, as well as CEBPD and HLA-F, were up-regulated in cysts mainly during the secretory phase. Over-expression of those genes leads to endometriosis and to poor reproductive and production of autoantibodies.

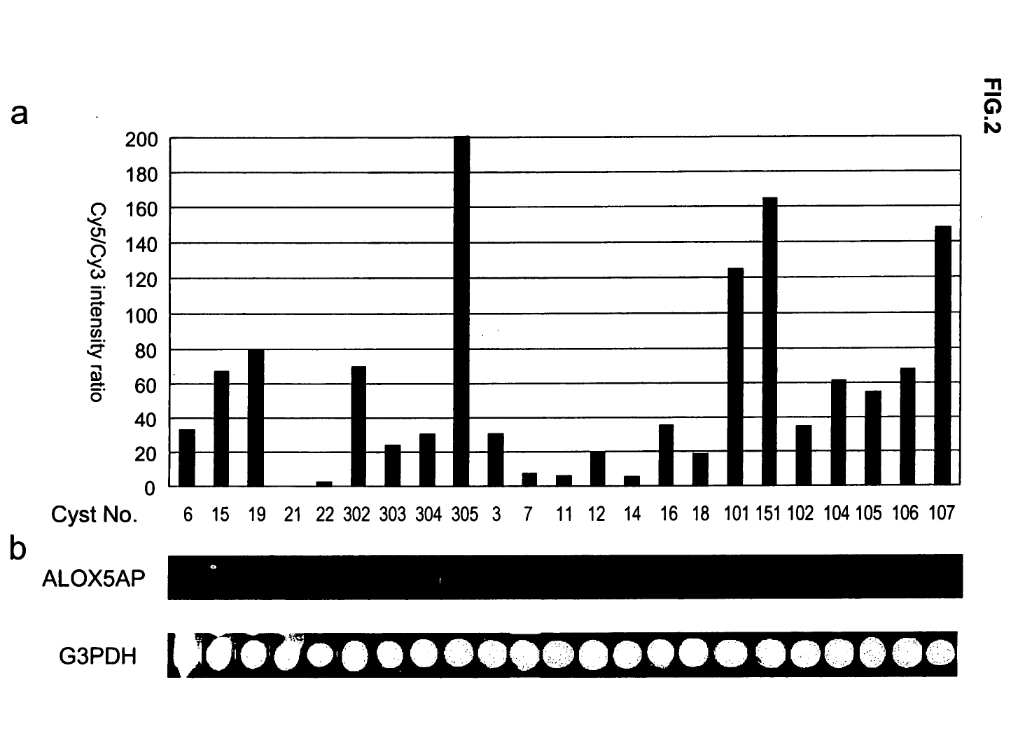

As a clinical entity, endometriosis appears to be an estrogen-dependent disease whose major symptoms are dysmenorrhea, dyspareunia, chronic pelvic pain and infertility. The results of the microarray analysis indicated concordance with these clinical features. For example, high expression of complement components indicates that pelvic pain results from severe inflammation in the endometriotic lesion. For example, the gene encoding ALOX5AP, which is required for leukotriene biosynthesis, was up-regulated in 22 of the 23 patients examined (FIG. 2). Progressive inflammation or adhesion induced by over-expression of these genes causes physical damage to tubes and ovaries, leading to infertility. Furthermore, local intraperitoneal inflammation generates ascites. Peritoneal fluid in patients with endometriosis reduces fertility by inhibiting fimbrial capability of the cumulus-oocyte complex, movement of sperm, and growth of the embryo.

One of the up-regulated genes is TGFBI. The product of this gene inhibits natural killer activity while inducing angiogenesis and proliferation of endometrial stromal cells. In addition, TGFBI significantly inhibits development of early mice embryos. Increased expression of TGFBI in endometriotic tissue is involved in the progression of endometriosis and in infertility.

The down-regulation of oviductal glycoprotein 1 (OVGP1), observed in endometrial cysts during both phases of the menstrual cycle, is also noteworthy in relation to infertility. OVGP1 plays a role in protecting the early embryo and the fallopian tube from extracellular environments that are potentially noxious, such as peritoneal fluid, retrograde reflux of menstrual fluid, microorganisms and spermatozoa. Lowered expression of OVGP1 therefore may contribute to infertility in patients with endometriosis.

Down-regulation of tumor suppressor TP53 in cysts from ten of the 14 patients was detected in the secretory menstrual phase and in cysts from six of the nine patients in the proliferative phase. TP53BP2 was also under-expressed in seven of the nine cases in the proliferative phase. Although it is considered a benign disorder, endometriosis exhibits tumor-like features that include cellular proliferation, cellular invasion and neoangiogenesis. A significantly higher risk of ovarian cancer is associated with endometriosis (standardized incidence ratio=1.9), especially among women with ovarian endometriosis of more than 10 years' duration (ratio=4.2) (Brinton et al. (1997) Am. J. Obstet. Gynecol. 176: 572-9).

The differential gene expression patterns described herein are useful to diagnose endometrioid and clear-cell carcinoma of the ovary. In one study about 39.2% of patients with ovarian clear-cell carcinomas and 21.2% of patients with ovarian endometrioid carcinomas were affected with ovarian endometriosis (Yoshikawa et al. (2000) Gynecol. Obest. Invest. 50: 11-7). Shimizu et al. have noted that clear-cell carcinomas of the ovary tend to show negative expression of p53 (Shimizu et al. (1999) Cancer 85: 669-77). The data described herein also indicate that under-expression of TP53 and/or TP53BP2 is involved in “malignant” endometriosis. Furthermore, GADD34, GADD45A and GADD45B, proteins associated with apoptosis (DeSmaele et al. (2001) Nature 414: 308-13), were also down-regulated in the cysts in spite of the fact that transcriptional levels of those genes tend to increase under stressful conditions of growth-arrest or following treatment with DNA-damaging agents. Moreover, PIG11, which generates or responds to oxidative stress and has a role in p53-dependent apoptosis, was down-regulated in 11 of the 14 cases examined in the secretory phase and in six of the nine cases in the proliferative phase. Hence, decreased expression of these genes in endometrial epithelial cells affects apoptotic signals and may be associated with the tumor-like character of this disease.

Synthesis of ribosomal proteins increases in response to estrogen (Knowles (1978) Biochem. J. 170: 181-3; Muller and Knowles (1984) FEBS Lett. 174: 253-7), and microarray analyses have shown up-regulation of ribosomal protein S23 (RPS23) in ectopic endometrium (Eyster et al. (2002) Fertil. Steril. 77: 38-42). The data described herein revealed up-regulation of RPS11 and RPL11 as well as RPS23. The increase in expression may be a consequence of increased estrogen levels in endometriotic tissue.

When cut-off value of 5.0 was applied for the signal-intensity ratios of Cy5/Cy3, 20 genes including four expressed-tag sequences (ESTs) were found to be “5 fold up-regulated” in at least 50% of the 23 cases examined (Table 7). The protocol selected 38 genes including 19 ESTs as “5 fold up-regulated” only in the proliferative phase (Table 8) and 35 genes including 10 ESTs only in the secretory phase (Table 9). Most of the genes listed in Table 8 were also over-expressed in the other phase (i.e., the secretory phase) and most of the genes listed in Table 9 were also over-expressed in the proliferative phase, but both in fewer than 50% of the cases. However, some genes were “5 fold up-regulated” in exclusively in one phase or the other: e.g., secreted frizzled-related protein 4 (SFRP4) and tissue factor pathway inhibitor 2 (TFPI2) were “5 fold up-regulated” specifically in the secretory phase.

The experiments also revealed down-regulation of numerous genes in the cysts; 36 genes, including 14 ESTs, were commonly under-expressed in more than 50% of the patients during either proliferative or secretory phases when the cut-off signal intensity ratio of Cy5/Cy3 was set to 0.2. Forty seven other genes were selected, including 13 ESTs, as being under-expressed only in the proliferative phase, and 276 genes including 156 ESTs only in the secretory phase.

Many of the “5 fold up-regulated” genes encode elements of the immune system; for example HLA-DRA, IGHG3, IGLλ, CIR and CD163 were all over-expressed throughout the menstrual cycle. In addition, genes encoding complement factors C3, BF, C1S and C2, as well as CEBPD and HLA-DQB1, were “5 fold up-regulated” in cysts mainly during the secretory phase.

As a clinical entity endometriosis appears to be an estrogen-dependent disease, the major symptoms of which are dysmenorrhea, dyspareunia, chronic pelvic pain and infertility. The results of the microarray analysis indicated concordance with these clinical features; e.g., high expression of complement components suggests that pelvic pain could result from severe inflammation in the endometriotic lesion. The correlation is supported by data indicating that the gene encoding ALOX5AP, which is required for leukotriene biosynthesis, was “5 fold up-regulated” in 21 of the 23 patients examined.

The nucleic acid sequences identified as “5 fold up-regulated” genes are useful as target of therapeutic agents for alleviating ovarian endometriosis or a predisposition to developing ovarian endometriosis.

### Example 5

### Confirmation of Expression of TFPI-2 and ITLN in Endometriosis by Semi-Quantitative RT-PCR

cDNA microarray was used to analyze gene-expression profiles of 23,040 genes in ovarian endometrial cysts from 23 patients (Arimoto et al. (2003) Int. J. Oncol. 22: 551-60). Among the up-regulated genes, the gene encoding the TFPI-2, which was found to be overexpressed in all of 9 informative cases whose signal intensities of the gene were higher than the cut-off in the secretory phase and down-regulated in more than 50% of the cases in the proliferative phase, was focused and selected for further study. In addition, ITLN, which was also up-regulated in 8 of 9 informative cases in the secretory phase and was up-regulated in all of 3 informative cases in the proliferative phase was also focused and selected for further studies. Furthermore, semi-quantitative RT-PCR analyses were performed to confirm the elevated expression of TFPI-2 in the secretory phase and that of ITLN throughout the menstrual cycle in endometriosis (FIG. 3).

### Example 6

### The Expression of the TFPI-2 and ITLN Proteins

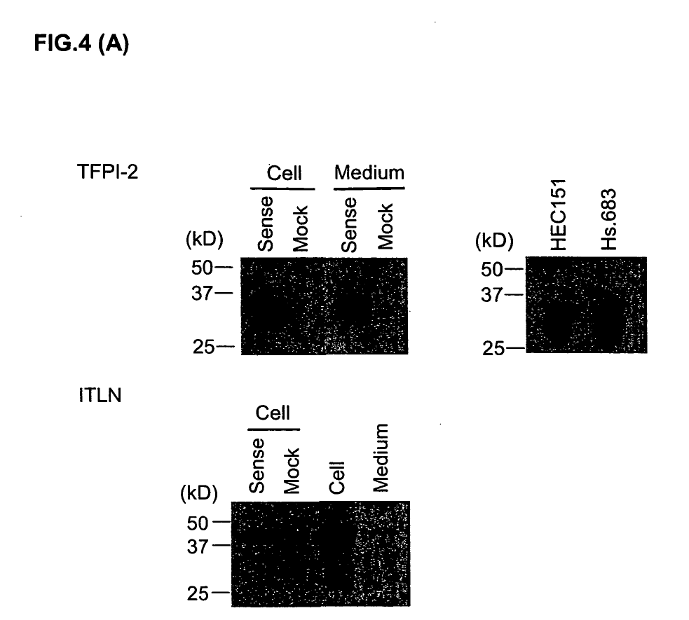

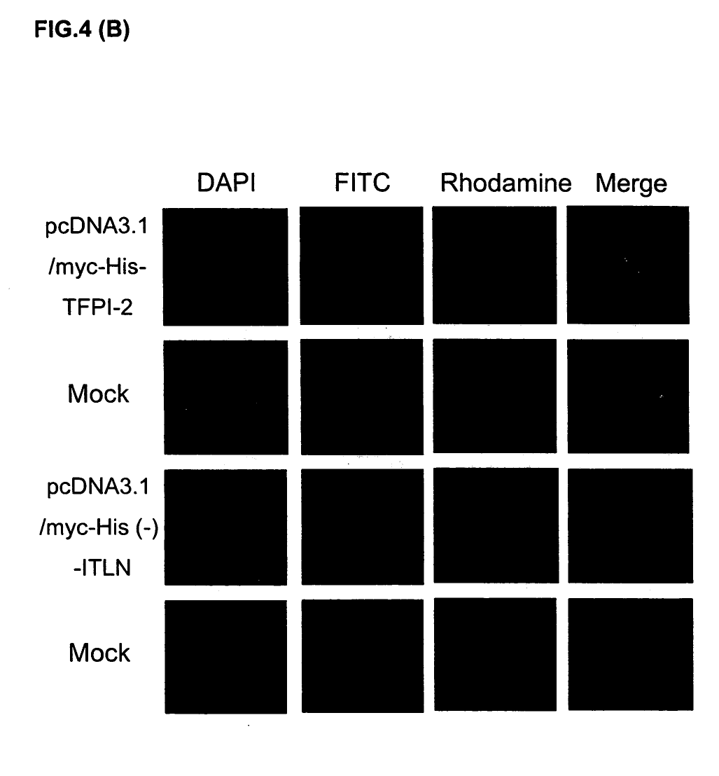

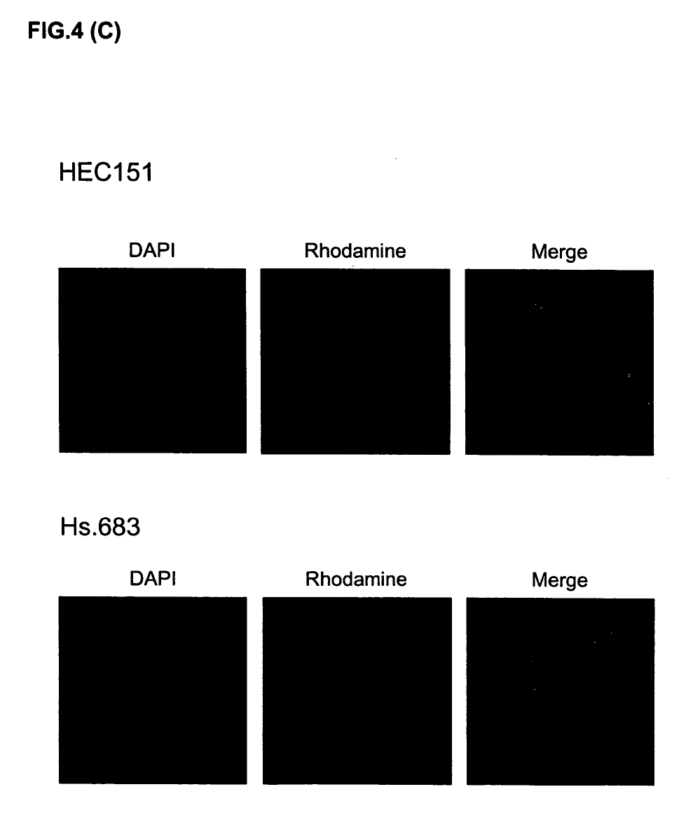

To investigate whether TFPI-2 and ITLN are secreted proteins as described previously, a population of mammalian cells that stably overexpress these proteins was established by transfecting pcDNA3.1 (−)-TFPI-2-myc-his or pcDNA3.1 (−)-ITLN-myc-his into COS7 cells. Expression and subcellular localization in some transformant was confirmed by Western blotting (FIG. 4A) and immunofluorescent staining (FIG. 4B). As described previously, prominent TFPI-2 triplet bands (approximately 33, 31 and 27 kDa) were observed for the TFPI-2 sense transformants with anti-TFPI-2 polyclonal antibody, and corresponding TFPI-2 proteins were also detected in culture medium (Sense), but not in the vector transfectants (Mock) (FIG. 4A; left panel). In addition, endogenous expression of the TFPI-2 protein in HEC-1 51 and Hs.683 cells detected by Western blotting (FIG. 4A, right panel) and immunofluorescent staining (FIG. 4C) with anti-TFPI-2 polyclonal antibody revealed that the TFPI-2 protein localizes in the cytoplasm like a secreted protein. Similarly, a 40 kDa single band of ITLN was also observed in culture medium of the ITLN sense transformants with anti-ITLN polyclonal antibody (FIG. 4A), and ITLN protein was observed to be localized in cytoplasm as well as the TFPI-2 (FIG. 4B). These results suggest that both TFPI-2 and ITLN are secreted proteins.

### Example 7

### Detection of TFPI-2 and ITLN Protein Expression in Endometrial Cysts and Normal Human Tissues by Immunohistochemistry

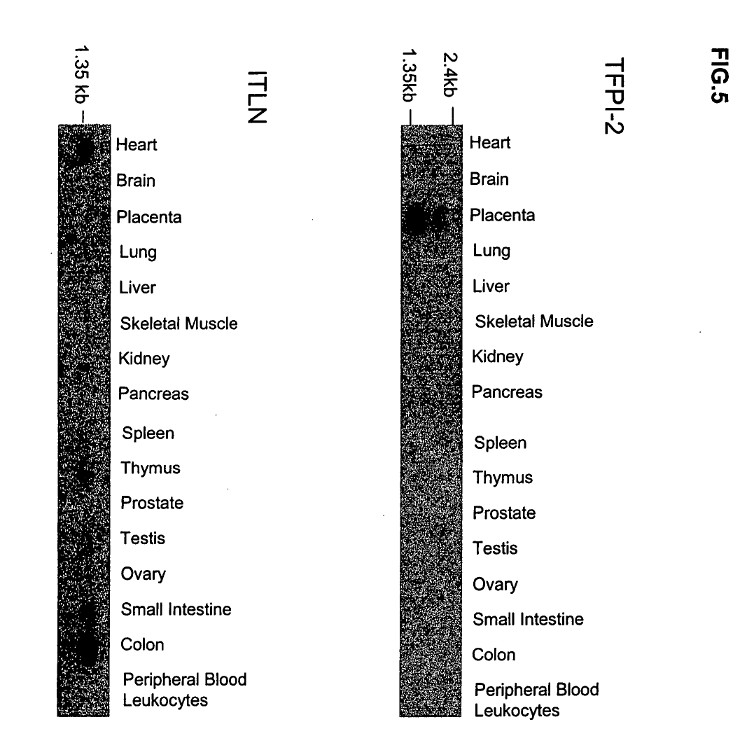

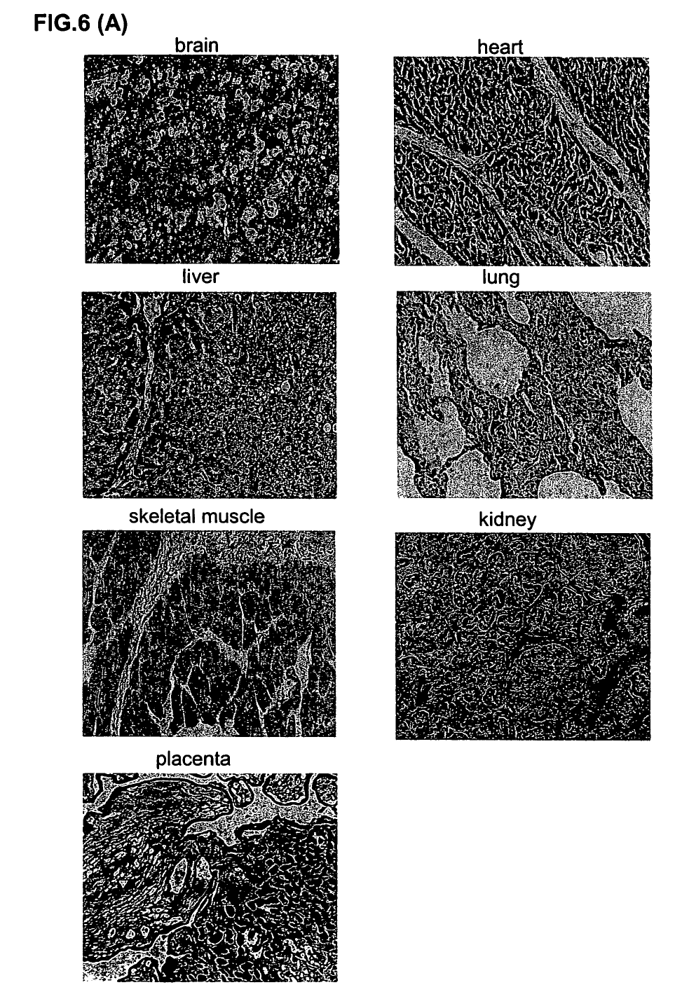

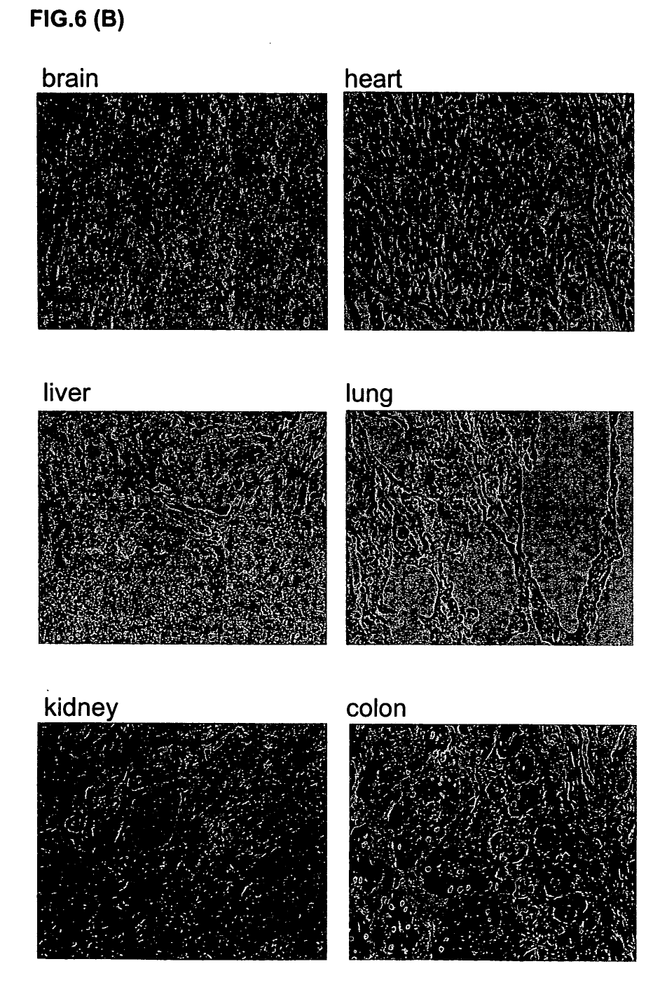

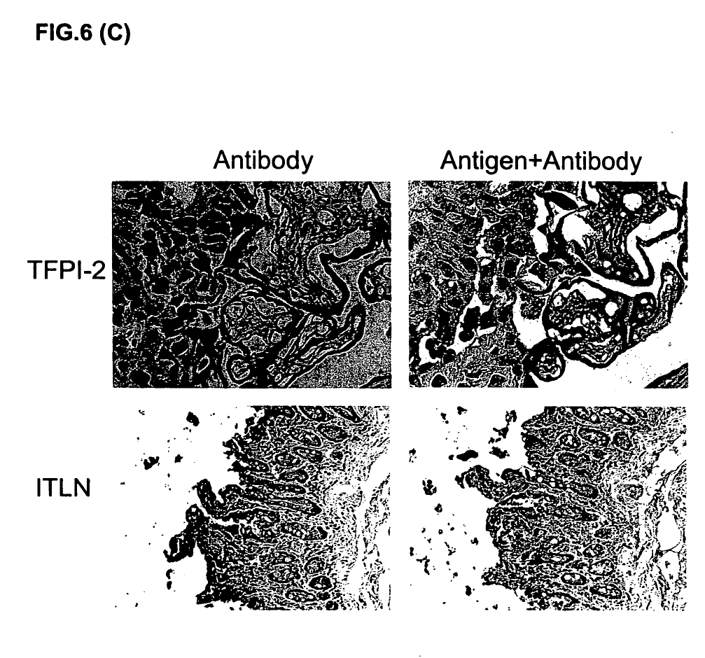

TFPI-2 was initially demonstrated to be specifically expressed in the placenta (FIG. 5, upper panel), and ITLN in the colon, and to a less degree in the heart, small intestine, thymus, testis and spleen (FIG. 5, lower panel) by Northern blot analysis. Next, immunohistochemical staining was performed on human normal tissues, and the TFPI-2 protein was demonstrated to be expressed in the syncytiotrophoblasts and decidual cells of placenta; weak expression was found in the cytoplasm of hepatocytes around the vein and cardiac muscle cells, and almost no staining (i.e., expression) was observed in brain, kidney, lung and skeletal muscle (FIG. 6A). On the other hand, positive staining for ITLN protein was observed in the basal cells of crypts and mucosal epithelial cells of colon and small intestine while the cytoplasm of cardiac muscle cells was stained weaker and brain, kidney, liver and lung was not stained at all (FIG. 6B).

Furthermore, to investigate whether TFPI-2 or ITLN antibodies can specifically recognize the corresponding proteins in placenta or small intestine, a cross-inhibition assay was performed. As a result, reduced staining of these cells showed that both polyclonal antibodies reacted specifically to their corresponding protein (FIG. 6C).

Moreover, expression of the TFPI-2 and ITLN proteins in the sections of endometrial cysts was investigated by immunohistochemistry. Positive staining for the protein was observed in epithelial cells of endometrial cysts by anti-TFPI-2 antibody or anti-ITLN-antibody (FIG. 7, left and middle panels), whereas no positive staining in the same enodometrial cysts tissues could be observed using anti-rabbit IgG as the negative control.

### Example 8

### Detection of the TFPI-2 Protein in Endometriotic Sera

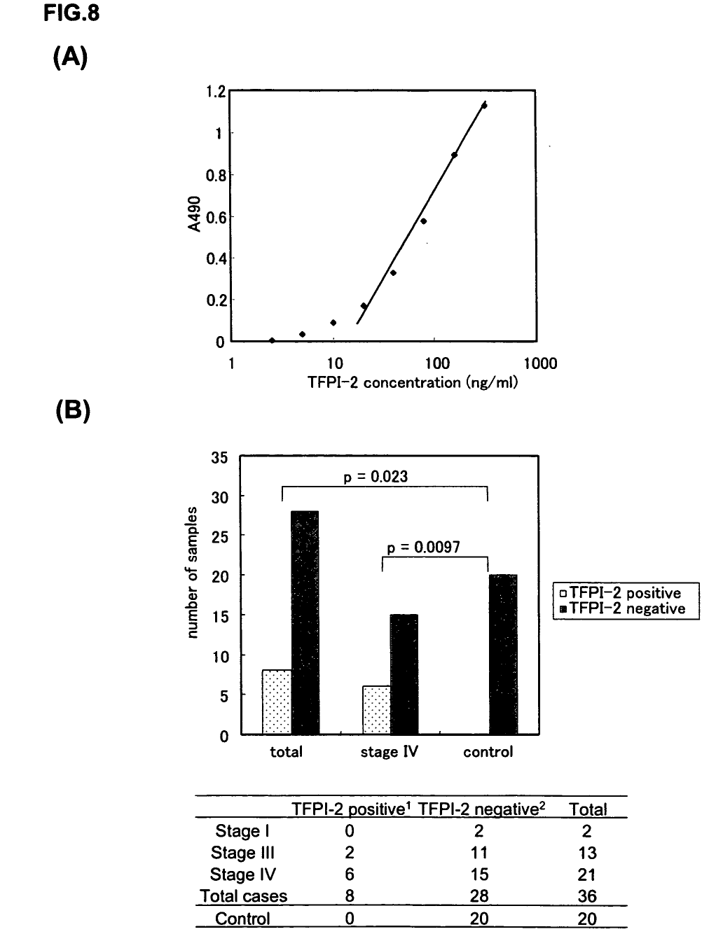

To further examine the possibility whether TFPI-2 serves as a diagnostic marker for endometriosis, an ELISA assay using specific TFPI-2 polyclonal antibodies was conducted to measure the concentration of the TFPI-2 protein in sera. The dose-response curve obtained according to the assay was linear over a range of 20 to 320 ng/ml of TFPI-2 (FIG. 8A). Using this standard curve, the concentration of the TFPI-2 protein in sera of patients with endometriosis detected by ELISA was calculated. It has been reported that the concentration of TFPI-2 in sera from both men and non-pregnant women was less than 1 ng/ml (Buztow et al. (1988) Clin. Chem. 34: 1591-3). Further, based on this reported concentration, the dose-response curve was confirmed to be linear within the range encompassing the concentration measured for eight healthy men and twelve healthy women studied as controls in this study. In eight of 36 endometrial serum samples, the level of TFPI-2 was >20 ng/ml (range 26.2-66.3 ng/ml, median 31.0 ng/ml), whereas that in all of 20 control samples was <20 ng/ml (FIG. 8B, p=0.023). In addition, the TFPI-2 protein was detected in six of 21 sera from rASRM stage IV patients (p=0.0097).

While the invention has been described in detail and with reference to specific embodiments thereof, it is to be understood that the foregoing description is exemplary and explanatory in nature and is intended to illustrate the invention and its preferred embodiments. Through routine experimentation, one skilled in the art will readily recognize that various changes and modifications can be made therein without departing from the spirit and scope of the invention. Thus, the invention is intended to be defined not by the above description, but by the following claims and their equivalents.

1. A method of diagnosing ovarian endometriosis or a predisposition of developing ovarian endometriosis in a subject, comprising determining the expression level of an ovarian endometriosis-associated gene in a subject-derived biological sample, wherein an increase or decrease of said expression level compared to a control level of said gene indicates that said subject suffers from or is at risk of developing ovarian endometriosis.

2. The method of claim 1, wherein said ovarian endometriosis-associated gene is selected from the group consisting of OEX 1-97 and 186-242, wherein an increase in said level compared to a normal control level indicates said subject suffers from or is at risk of developing ovarian endometriosis.

3. The method of claim 2, wherein said increase is at least 10% greater than said normal control level.

4. The method of claim 1, wherein said ovarian endometriosis-associated gene is selected from the group consisting of OEX 98-185, wherein a decrease in said level compared to a normal control level indicates said subject suffers from or is at risk of developing ovarian endometriosis.

5. The method of claim 4, wherein said decrease is at least 10% lower than said normal control level.

6. The method of claim 1, wherein said method further comprises determining the expression level of a plurality of the ovarian endometriosis-associated genes.

7. The method of claim 1, wherein the expression level is determined by any one method selected from the group consisting of:
(a) detecting the mRNA of an ovarian endometriosis-associated gene;
(b) detecting the protein encoded by an ovarian endometriosis-associated gene; and
(c) detecting the biological activity of the protein encoded by an ovarian endometriosis-associated gene.

8. The method of claim 7, wherein said expression level is determined by detecting hybridization of an ovarian endometriosis-associated gene probe to a gene transcript in said subject-derived biological sample.

9. The method of claim 8, wherein said hybridization step is carried out on a DNA array.

10. The method of claim 1, wherein said biological sample is a tissue sample and comprises an epithelial cell.

11. The method of claim 1, wherein said biological sample is a tissue sample comprising an endometrial cyst cell.

12. The method of claim 11, wherein said biological sample is a tissue sample comprising an epithelial cell from an endometrial cyst.

13. An ovarian endometriosis reference expression profile, comprising a pattern of gene expression of two or more genes selected from the group consisting of OEX 1-242.

14. An ovarian endometriosis reference expression profile, comprising a pattern of gene expression of two or more genes selected from the group consisting of OEX 1-97 and 86-242.

15. An ovarian endometriosis reference expression profile, comprising a pattern of gene expression of two or more genes selected from the group consisting of OEX 98-185.

16. A kit comprising one or more detection reagents which binds to one or more nucleic acid sequences selected from the group consisting of OEX 1-242.

17. An array comprising one or more nucleic acids which bind to one or more nucleic acid sequences selected from the group consisting of OEX 1-242.

18. A method of diagnosing ovarian endometriosis in a subject, comprising the steps of:
(1) determining the expression level of a TFPI-2 protein in a subject-derived biological sample, and (2) comparing the expression level determined in step (1) with a control level, wherein an increase in expression level as compared to a normal control level indicates that said subject suffers from ovarian endometriosis and a similarity of said expression level as compared to a disease control level indicates that said subject suffers from ovarian endometriosis.

19. The method of claim 18, wherein the expression level is determined by immunoassay.

20. The method of claim 19, wherein the immunoassay is ELISA.

21. The method of claim 20, wherein a polyclonal antibody and a monoclonal antibody against the TFPI-2 protein is used as the labeled antibody and immobilized antibody, respectively, for ELISA.

22. The method of claim 18, wherein the biological sample is blood or a blood-derived sample.

23. A kit for diagnosing ovarian endometriosis comprising one or more antibodies binding to a TFPI-2 protein.

24. The kit of claim 23, which comprises (1) a detectably labeled antibody binding to the TFPI-2 protein, and (2) an antibody binding to the TFPI-2 protein that is immobilized on a solid matrix.

25. The kit of claim 24, wherein the detectably labeled antibody is polyclonal and the antibody immobilized on a solid matrix is monoclonal.

26. The kit of claim 23, which comprises (1) a detectably labeled antibody binding to the TFPI-2 protein, (2) an antibody binding to the TFPI-2 protein which may be immobilized on a matrix, and (3) a reagent for immobilizing the antibody of (2) on a matrix.

27. The kit of claim 26, wherein the detectably labeled antibody is polyclonal and the antibody that may be immobilized on a solid matrix is monoclonal.

28. The kit of claim 23, wherein the kit further comprises either or both of normal control sample and endometriosis control sample.
